(19) United States
(12) Patent Application Publication
(10) Pub. No.: US 2011/0143958 A1
(43) Pub. Date: Jun. 16, 2011

(54) Methods of Identifying Modulators of Ubiquitin Ligases

(76) Inventors: Jeffrey Gordon Marblestone, Langhorne, PA (US); Benjamin Nicholson, Merion Station, PA (US); Suresh Kumar Kizhakkethil-George, Downingtown, PA (US)

(21) Appl. No.: 13/055,042

(22) PCT Filed: Jul. 23, 2009
(86) PCT No.: PCT/US09/51538

§ 371 Date: Feb. 24, 2011

Related U.S. Application Data
(60) Provisional application No. 61/083,756, filed on Jul. 25, 2008.

(51) Int. Cl. C40B 30/04; G01N 33/53

(52) U.S. Cl. 506/9; 435/7.8

(57) ABSTRACT

Methods for identifying ubiquitin ligases and ubiquitin ligase modulators are disclosed. The methods comprise combining the components of a ubiquitylation reaction and using proteins that contain motifs that recognize ubiquitylated sites in the detection of ubiquitylation.

### BACKGROUND TECHNOLOGY

Ubiquitin is a post-translation, modifying protein that is covalently attached to lysine side-chains of a variety of target proteins. Given the variety of target proteins, ubiquitin attachment plays a role in a number of different biological processes. Aberrations in ubiquitin attachment have the potential to result in a variety of conditions, diseases, and/or syndromes. Of therapeutic interest are ubiquitin ligases, enzymes that attach ubiquitin to target proteins, in part because of their numerosity (hundreds are predicted in the human proteome) and specificity (each ligase has specificity for a target protein). Furthermore, a related group of proteins, ubiquitin-like protein modifiers, and their corresponding ligases have also been implicated in a variety of biological functions. The therapeutic potential in modulating these ligases has yet to be fully realized. One reason may be the lack of facile methods to identify and characterize these ligases and screen compounds that modulate their activity.

### SUMMARY

The present invention features methods of identifying ubiquitin ligases and modulators of the same by taking advantage of the ability of certain motifs, described as ubiquitin-adhering motifs herein, to bind ubiquitin (“Ub”) and ubiquitin-like proteins (“Ubl”).

Certain embodiments relate to methods of identifying a ubiquitin ligase by combining a putative ubiquitin ligase, ubiquitin-adhering motif-containing protein, ubiquitin-activating enzyme, ubiquitin-conjugating enzyme, Ub/Ubl, ATP, and substrate for ubiquitin ligase; measuring the amount of Ub/Ubl bound to the substrate; comparing the amount of Ub/Ubl bound to the substrate in the presence of the ubiquitin ligase to the amount of Ub/Ubl bound to the substrate in the absence of the ubiquitin ligase, whereby an increase in amount of Ub/Ubl bound to the substrate indicates that the putative ubiquitin ligase is a ubiquitin ligase. In certain embodiments it may be advantageous that the ubiquitin-adhering motif-containing protein be bound to a solid support. There are a variety of suitable ubiquitin-adhering motifs, such as UBA, UIM, CUE, NZF, UEV, GLUE, MIU and/or GAT. In certain embodiments it may be advantageous that an ubiquitin-adhering motif-containing protein contain more than one more than one ubiquitin-adhearing motif, preferably link (or fused) together. In situations in which an ubiquitin-adhering motif-containing protein contains more than one ubiquitin-adhering motif, the motifs may be the same or different. Suitable methods for detecting whether ubiquitin or a ubiquitin-like protein have been bound to a substrate include antibody technology, fluorescence polarization, fluorescence intensity, fluorescence resonance transfer, chromogenicity, and/or luminescence. Various characteristics of the ubiquitin ligase may also be determined using a concentration gradient of Ub/Ubl, substrate, ubiquitin ligase, or combination thereof.

Certain embodiments relate to identifying a ubiquitin ligase modulator by measuring the amount of Ub/Ubl bound to a ubiquitin ligase substrate and may be accomplished by combining ubiquitin ligase, ubiquitin-adhering motif-containing protein, ubiquitin-activating enzyme, ubiquitin-conjugating enzyme, Ub/Ubl, ATP, and substrate for ubiquitin ligase, in the presence of a candidate modulator; measuring the amount of Ub/Ubl bound to the substrate; and comparing the amount of Ub/Ubl bound to the substrate in the presence of the candidate modulator to the amount of Ub/Ubl bound to the substrate in the absence of the candidate modulator, whereby the difference in amount of Ub/Ubl bound to the substrate indicates that the candidate modulator is a ubiquitin ligase modulator. The candidate modulator is a ubiquitin ligase activator where there is a positive difference in amount of Ub/Ubl bound to the substrate. The candidate modulator is a ubiquitin ligase inhibitor where there is a negative difference in the amount of Ub/Ubl bound to the substrate. In certain embodiments it may be advantageous that the ubiquitin-adhering motif-containing protein be bound to a solid support. There are a variety of suitable ubiquitin-adhering motifs, such as UBA, UIM, CUE, NZF, UEV, GLUE, MIU and/or GAT. In certain embodiments it may be advantageous that an ubiquitin-adhering motif-containing protein contain more than one more than one ubiquitin-adhearing motif, preferably link (or fused) together. In situations in which an ubiquitin-adhering motif-containing protein contains more than one ubiquitin-adhering motif, the motifs may be the same or different. Suitable methods for detecting whether ubiquitin or a ubiquitin-like protein have been bound to a substrate include antibody technology, fluorescence polarization, fluorescence intensity, fluorescence resonance transfer, chromogenicity, and/or luminescence.

Certain embodiments feature identifying a ubiquitin ligase modulator by measuring the amount of Ub/Ubl bound to the ubiquitin ligase, and may be accomplished by combining ubiquitin ligase, ubiquitin-adhering motif-containing protein, ubiquitin-activating enzyme, ubiquitin-conjugating enzyme, Ub/Ubl, and ATP, in the presence of a candidate modulator; measuring the amount of Ub/Ubl bound to ubiquitin ligase; and comparing the amount of Ub/Ubl bound to ubiquitin ligase in the presence of the candidate modulator to the amount of ubiquitin bound to ubiquitin ligase in the absence of the candidate modulator, whereby the difference in amount of Ub/Ubl bound to ubiquitin ligase indicates that the candidate modulator is a ubiquitin ligase modulator. The candidate modulator is a ubiquitin ligase activator where there is a positive difference in amount of Ub/Ubl bound to the ligase. The candidate modulator is a ubiquitin ligase inhibitor where there is a negative difference in the amount of Ub/Ubl bound to the ligase. In certain embodiments it may be advantageous that the ubiquitin-adhering motif-containing protein be bound to a solid support. There are a variety of suitable ubiquitin-adhering motifs, such as UBA, UIM, CUE, NZF, UEV, GLUE, MIU and/or GAT. In certain embodiments it may be advantageous that an ubiquitin-adhering motif-containing protein contain more than one more than one ubiquitin-adhearing motif, preferably link (or fused) together. In situations in which an ubiquitin-adhering motif-containing protein contains more than one ubiquitin-adhering motif, the motifs may be the same or different. Suitable methods for detecting whether ubiquitin or a ubiquitin-like protein have been bound to a ligase include antibody technology, fluorescence polarization, fluorescence intensity, fluorescence resonance transfer, chromogenicity, and/or luminescence.

Other embodiments feature identifying compounds for treating a disease associated with aberrant ubiquitylation of a ubiquitin ligase substrate, and may be accomplished by combining ubiquitin ligase, ubiquitin-adhering motif-containing protein, ubiquitin-activating enzyme, ubiquitin-conjugating enzyme, Ub/Ubl, and ATP, and substrate for ubiquitin ligase, in the presence of a candidate modulator; measuring the amount of Ub/Ubl bound to the substrate; and comparing the amount of Ub/Ubl bound to the substrate in the presence of the candidate modulator to the amount of ubiquitin bound to ubiquitin ligase in the absence of the candidate modulator, whereby the difference in amount of Ub/Ubl bound to the substrate indicates that the candidate modulator is a ubiquitin ligase modulator and may be suitable for treating the disease. In certain embodiments the ubiquitin ligase is Parkin and the disease is Parkinson's disease. In other embodiments the ubiquitin ligase is MDM2 and the disease is cancer. In further embodiments the ubiquitin ligase is MuRF1 and the disease is muscular degeneration. The candidate modulator is a ubiquitin ligase activator where there is a positive difference in amount of Ub/Ubl bound to the substrate. The candidate modulator is a ubiquitin ligase inhibitor where there is a negative difference in the amount of Ub/Ubl bound to the substrate. In certain embodiments it may be advantageous that the ubiquitin-adhering motif-containing protein be bound to a solid support. There are a variety of suitable ubiquitin-adhering motifs, such as UBA, UIM, CUE, NZF, UEV, GLUE, MIU and/or GAT. In certain embodiments it may be advantageous that an ubiquitin-adhering motif-containing protein contain more than one more than one ubiquitin-adhearing motif, preferably link (or fused) together. In situations in which an ubiquitin-adhering motif-containing protein contains more than one ubiquitin-adhering motif, the motifs may be the same or different. Suitable methods for detecting whether ubiquitin or a ubiquitin-like protein have been bound to a substrate include antibody technology, fluorescence polarization, fluorescence intensity, fluorescence resonance transfer, chromogenicity, and/or luminescence.

Further embodiments relate to methods of identifying a compound for treating a disease associated with aberrant ubiquitylation of a ubiquitin ligase, and may be accomplished by combining ubiquitin ligase, ubiquitin-adhering motif-containing protein, ubiquitin-activating enzyme, ubiquitin-conjugating enzyme, Ub/Ubl, and ATP, in the presence of a candidate modulator; measuring the amount of Ub/Ubl bound to ubiquitin ligase; and comparing the amount of Ub/Ubl bound to ubiquitin ligase in the presence of the candidate modulator to the amount of ubiquitin bound to ubiquitin ligase in the absence of the candidate modulator, whereby the difference in amount of Ub/Ubl bound to ubiquitin ligase indicates that the candidate modulator is a ubiquitin ligase modulator and may be suitable for treatment of the disease. In certain embodiments the ubiquitin ligase is Parkin and the disease is Parkinson's disease. In other embodiments the ubiquitin ligase is MDM2 and the disease is cancer. In further embodiments the ubiquitin ligase is MuRF1 and the disease is muscular degeneration. The candidate modulator is a ubiquitin ligase activator where there is a positive difference in amount of Ub/Ubl bound to the ligase. The candidate modulator is a ubiquitin ligase inhibitor where there is a negative difference in the amount of Ub/Ubl bound to the ligase. In certain embodiments it may be advantageous that the ubiquitin-adhering motif-containing protein be bound to a solid support. There are a variety of suitable ubiquitin-adhering motifs, such as UBA, UIM, CUE, NZF, UEV, GLUE, MIU and/or GAT. In certain embodiments it may be advantageous that an ubiquitin-adhering motif-containing protein contain more than one more than one ubiquitin-adhearing motif, preferably link (or fused) together. In situations in which an ubiquitin-adhering motif-containing protein contains more than one ubiquitin-adhering motif, the motifs may be the same or different. Suitable methods for detecting whether ubiquitin or a ubiquitin-like protein have been bound to a substrate include using antibodies, fluorescence polarization, fluorescence intensity, fluorescence resonance transfer, chromogenicity, and/or luminescence.

Certain embodiments feature methods of selecting a ubiquitin ligase modulator using step-wise screening strategies for altered ubiquitylation of a substrate. These methods may be accomplished by providing an array of wells, whereby each well contains ubiquitin-adhering motif-containing protein, ubiquitin-activating enzyme, ubiquitin-conjugating enzyme, Ub/Ubl, ATP, a plurality of ubiquitin ligases, and a ubiquitin ligase substrate in the presence of a candidate modulator; measuring the amount of Ub/Ubl bound to the substrate; and comparing the amount of Ub/Ubl bound to the substrate in the presence of the candidate modulator to the amount of Ub/Ubl bound to the substrate in the absence of the candidate modulator, whereby the difference in amount of Ub/Ubl bound to the substrate indicates that the candidate modulator is a ubiquitin ligase modulator. These steps may be repeated such that the plurality of ubiquitin ligases are substituted with a subset of ubiquitin ligases from those wells having a difference in amount of ubiquitin bound in the presence of the candidate modulator until one arrives at a well containing one ubiquitin ligase that displays modulated activity in the presence of the modulator. The candidate modulator is a ubiquitin ligase activator where there is a positive difference in the amount of Ub/Ubl bound to the substrate. The candidate modulator is a ubiquitin ligase inhibitor where there is a negative difference in the amount of Ub/Ubl bound to the substrate. In certain embodiments it may be advantageous that the ubiquitin-adhering motif-containing protein be bound to a solid support. There are a variety of suitable ubiquitin-adhering motifs, such as UBA, UIM, CUE, NZF, UEV, GLUE, MIU and/or GAT. In certain embodiments it may be advantageous that an ubiquitin-adhering motif-containing protein contain more than one more than one ubiquitin-adhearing motif, preferably link (or fused) together. In situations in which an ubiquitin-adhering motif-containing protein contains more than one ubiquitin-adhering motif, the motifs may be the same or different. Suitable methods for detecting whether ubiquitin or a ubiquitin-like protein have been bound to a substrate include using antibodies, fluorescence polarization, fluorescence intensity, fluorescence resonance transfer, chromogenicity, and/or luminescence.

Certain embodiments feature methods of selecting a ubiquitin ligase modulator using step-wise screening strategies for altered ubiquitylation of a ubiquitin ligase. These methods may be accomplished providing an array of wells, whereby each well contains ubiquitin-adhering motif-containing protein, ubiquitin-activating enzyme, ubiquitin-conjugating enzyme, Ub/Ubl, ATP, a plurality of ubiquitin ligases, in the presence of a candidate modulator; measuring the amount of Ub/Ubl bound to the ubiquitin ligases; and comparing the amount of Ub/Ubl bound to the ubiquitin ligases in the presence of the candidate modulator to the amount of Ub/Ubl bound to the ubiquitin ligases in the absence of the candidate modulator, whereby the difference in amount of Ub/Ubl bound to the ubiquitin ligases indicates that the candidate modulator is a ubiquitin ligase modulator. These steps may be repeated such that the plurality of ubiquitin ligases are substituted with a subset of ubiquitin ligases from those wells having a difference in amount of ubiquitin bound in the presence of the candidate modulator until one arrives at a well containing one ubiquitin ligase that displays modulated activity in the presence of the modulator. The candidate modulator is a ubiquitin ligase activator where there is a positive difference in amount of Ub/Ubl bound to the ligase. The candidate modulator is a ubiquitin ligase inhibitor where there is a negative difference in the amount of Ub/Ubl bound to the ligase. In certain embodiments it may be advantageous that the ubiquitin-adhering motif-containing protein be bound to a solid support. There are a variety of suitable ubiquitin-adhering motifs, such as UBA, UIM, CUE, NZF, UEV, GLUE, MIU and/or GAT. In certain embodiments it may be advantageous that an ubiquitin-adhering motif-containing protein contain more than one more than one ubiquitin-adhearing motif, preferably link (or fused) together. In situations in which an ubiquitin-adhering motif-containing protein contains more than one ubiquitin-adhering motif, the motifs may be the same or different. Suitable methods for detecting whether ubiquitin or a ubiquitin-like protein have been bound to a substrate include antibody technology, fluorescence polarization, fluorescence intensity, fluorescence resonance transfer, chromogenicity, and/or luminescence.

### BRIEF DESCRIPTION OF THE DRAWINGS

FIG. 1 is the amino acid sequence of 6×His-SUMO-UBA2 (SEQ ID NO:30) (FIG. 1A) & 6×His-SUMOG2C-UBA1 (SEQ ID NO:31) (FIG. 1B) with the respective UBA domain sequences underlined.

FIG. 2 is an image of a Western Blot of various fractions of a CARP2 ubiquitylation reaction collected after passing through a SUMO-UBA2 column, which binds autoubiquitylated E3. An anti-ubiquitin antibody was used to detect the presence of ubiquitin.

FIG. 3A is a graph depicting the binding of polyubiquitin to SUMO-UBA2-coated plates. High-binding modular plates were coated with 6× His-SUMO-UBA2, incubated with mono- or poly-ubiquitin, and incubated with antibodies to detect bound product. The UBA2-coated plate specifically binds polyubiquitin.

FIG. 3B is a graph depicting the affinity of UBA2-coated plates for K48 and K63 polyubiquitin chains.

FIG. 4A is a graph depicting the binding of E3 reactions to SUMO-UBA2-coated plates. The ubiquitylation reactions were conducted where individual key components (−E3, −E2, −E1, and −Ub) of the reaction were absent. Only when all of these components are present ubiquitylation occurs in a time dependent manner.

FIG. 4B is a graph depicting the binding of E3 reactions with several E3 ligases in the presence of wild type or mutant ubiquitin (K48 and K63) to SUMO-UBA2-coated plates.

FIG. 5A is a graph depicting concentration dependency of an E3 (SUMO-CARP2) ubiquitylation assay.

FIG. 5B is a graph depicting concentration dependency of an E3 (GST-Praja1) ubiquitylation assay.

FIG. 6A is a graph depicting the IC₅₀ of three inhibitors, NEM, iodoacetamide, and Ubistatin A, of the ubiquitylation assay using 6× His-SUMO-CARP2 ligase.

FIG. 6B is a graph depicting the Z′ data distribution of a CARP2 ligase assay.

FIG. 7A is an image of an electrophoretic gel in which various fractions collected during the purification of 6× His-SUMO-UBA1 expressed in E. coli BL21 were separated and stained with coomassie brilliant blue.

FIG. 7B is a graph depicting a 6× His-SUMO-GFP-CARP2 ubiquitylation assay that was performed in a 96-well plate coated with 6× His-SUMO^{G2C}-UBA1. “No ligase” and “No Ub” are samples in which 6× His-SUMO-GFP-CARP2 or ubiquitin was omitted from the reaction.

FIG. 7C is an image of an electrophoretic gel in which fractions from a PD10 column were separated and visualized with a fluorescence imager. The 6× His-SUMO^{G2C}-UBA1 ubiquitylation reaction was performed as described in the Examples and the unreacted 5′-Iodoacetamido Fluorescein (“5′-IAF”) was removed using a PD10 desalting column.

FIG. 7D is an image of an electrophoretic gel in which fractions from a PD10 column were separated and stained with Coomassie brilliant blue. The 6× His-SUMO^{G2C}-UBA1 ubiquitylation reaction was performed as described in the methods and the unreacted 5′-IAF was removed using a PD10 desalting column.

FIG. 8 is a graph depicting the detection of fluorescently labeled 6× His-SUMO^{G2C}-UBA1. Polyubiquitylated CARP2 was detected using 500 nM of 5′-IAF labeled 6× His-SUMO^{G2C}-UBA1.

FIG. 9A is a graph depicting the effect of NEM on ubiquitylation. The E3 ubiquitylation assay was performed after pre-incubating the enzymes with various concentrations of NEM for 30 minutes at room temperature prior to the addition of ubiquitin/ATP to initiate the E3 reaction. The assay was analyzed using a fluorescence plate reader to detect the binding of an anti-ubiquitin antibody.

FIG. 9B is an image of an electrophoretic gel from the E3 ubiquitylation assay described in FIG. 9A.

FIG. 9C is a graph depicting the effect of Ubistatin A on ubiquitylation. The E3 ubiquitylation assay was performed after pre-incubating the enzymes with different concentrations of Ubistatin A for 30 minutes at room temperature prior to the addition of ubiquitin/ATP to initiate the E3 reaction. The assay was analyzed using a fluorescence plate reader to detect the binding of an anti-ubiquitin antibody.

FIG. 9D is an image of an electrophoretic gel from the E3 ubiquitylation assay described in FIG. 9C.

FIG. 10 is a graph depicting the ubiquitylation assay using 5-IAF labeled SUMO-CARP2, in which 5′-IAF-labeled 6× His-SUMO-CARP2 and 6× His-GFP-CARP2 were incubated in a reaction in the presence or absence of El. Mean RFU values were plotted.

### DETAILED DESCRIPTION OF ILLUSTRATIVE EMBODIMENTS

### Components of the Ubiquitin System

As used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “ubiquitin” (or “Ub”) and “ubiquitin-like protein modifiers” (or “Ubl”) refer to a family of proteins that share a characteristic β-grasp fold (“the ubiquitin fold”) and may be conjugated to proteins or lipids via their C-terminus. “Ubiquitin” is composed of 76 amino acids, having a molecular mass of about 8.5 kDa and high conservation among eukaryotes. The human ubiquitin sequence is: MQIFVKTLTGKTITLEVEPSDTIENVKAKIQDKEGIPPDQQRLIFAGKQLEDGRTLSDYNI QKESTLHLVLRLRGG (SEQ ID NO: 1). The C-terminal Glycine₇₅-Glycine₇₆ residues of ubiquitin are the key residues that function in the diverse chemistry of ubiquitin reactions. “Ub” and “Ubl” are also used herein to refer to fragments and/or variants thereof that participate in the ubiquitin system as described herein.

Like Ub, Ubls are also small proteins that may be conjugated to proteins or lipids via their C-terminus. Preferred embodiments of Ubls include, but are not limited to, Small Ubiquitin-related Modifier 1 (“SUMO1,” UniProt/Swiss-Prot Identifier: P63165); Small Ubiquitin-related Modifier 2 “SUMO2,” UniProt/Swiss-Prot Identifier: P61956) Small Ubiquitin-related Modifier 3 (“SUMO3,” UniProt/Swiss-Prot Identifier: P55854), NEDD8 (also known as Rub1; UniProt/Swiss-Prot Identifier: Q15843), FAT10 (also known as Ubiquitin D; UniProt/Swiss-Prot Identifier: O15205), Interferon-induced 15 kDa protein (“ISG15,” UniProt/Swiss-Prot Identifier: P05161), Ubiquitin-related modifier 1 homolog (“Urm1,” UniProt/Swiss-Prot Identifier: Q9BTM9); Ubiquitin-fold modifier 1 (“Ufm1,” UniProt/Swiss-Prot Identifier: P61960); Fau Ubiquitin-Like Protein 1 (“FUB1,” UniProt/Swiss-Prot Identifier: P35544); Ubiquitin-like protein 5 (“Ubl5,” also known as Hub1; UniProt/Swiss-Prot Identifier: Q9BZL1); Autophagy-related protein 8 (“Atg8,” also known as APG8 and AUT7, UniProt/Swiss-Prot Identifier: P38182), and Autophagy-related protein 12 (“Atg12,” also known as APG12 and APG12L; UniProt/Swiss-Prot Identifier: O94817). Certain embodiments use fragments and/or variants of these Ubls that participate in the ubiquitin system as described herein.

The ubiquitin system involves attaching (or removing) Ub to other components of the ubiquitin system and target proteins or lipids (or substrates) which effectuate a variety of biological processes. Central to the system is “ubiquitylation” (also known as ubiquitination), which refers to the cascade that results in the attachment one or more Ub to a substrate. The ubiquitylation cascade is generally understood to involve three phases: (1) activation, (2) transfer, and (3) recognition. Activation refers to a two-step reaction in which a Ub becomes attached to an E1 ubiquitin-activating enzyme (“ubiquitin-activating enzyme” or “E1”). In the first step of activation, E1 forms a ubiquitin-adenylate intermediate using ATP. In the second step of activation, Ub is bound to the E1 active site by a thioester bond between the Ub C-terminal carboxyl group and the E1 cysteine sulfhydryl group. Transfer refers to the exchange of Ub from E1 to the active site cysteine of a ubiquitin-conjugating enzyme E2 (or “ubiquitin-conjugating enzyme,” “E2,” or “ubiquitin carrier enzyme”) via a trans(thio)esterification reaction. Recognition involves a E3 ubiquitin-protein ligase (or “ubiquitin ligase” or “E3”), either enzymatically or acting as a scaffold for the E2, facilitating the transfer of Ub from E2 and a lysine on a substrate. After the linkage between Ub and substrate, additional Ub proteins can be conjugated to the previous ubiquitin forming a ubiquitin-chain through an amide (isopeptide) bond. The C-terminus of one ubiquitin moiety is attached to one of seven lysine residues (K6, K11, K27, K29, K33, K48 or K63) on an adjacent ubiquitin.

Similar to Ub, Ubls are activated, transferred, and recognized in a cascade analogous to ubiquitylation. Accordingly, the “ubiquitin system” and the “ubiquitin system components” should be understood to include Ubls and the corresponding E1, E2, and E3 enzymes that attach Ubls to substrates. “Ubiquitylation” should be understood herein to also include the Ubl cascade and resulting attachment of Ubls to a substrate. Also similar to Ub, multiple Ubls may be attached to a substrate. Accordingly, it should be understood that references to “ubiquitin-activating enzyme,” “ubiquitin-conjugating enzyme,” “ubiquitin ligase,” and the like are also intended to encompass and be used interchangeably with “ubiquitin-like protein modifier-activating enzyme,” “ubiquitin-like protein modifier-conjugating enzyme,” “ubiquitin-like protein modifier ligase,” and the like.

“Ubiquitin-activating enzyme” or “E1” refers to the family of enzymes that, as described above, are involved the activation of Ub/Ubl as part of the first step in the ubiquitylation pathway. Many proteins having E1 activity have been identified and reported in the scientific literature and cataloged on databases, such as the European Bioinformatics Institute's (“EBI”) Gene Ontology Annotation (“GOA”) Database that provides annotations to proteins in the UniProt Knowledgebase (UniProtKB) and Interation Protein Index (IPI). GOA also contains multi-species information from other databases, such as Ensemble and National Center for Biotechnology Information (“NCBI”). Access to this database is publicly available at http://www.ebi.ac.uk/ego/. Proteins having E1 activity can be found using the GO term identifiers GO:0008641; GO:0019782; GO:0004839; GO:0019781 and the “Protein Annotation” tab for the particular sequence identifiers. Those skilled in the art would also appreciate that the retrieved proteins can be limited to any individual species. In the case of human proteins, one would use the NCBI taxonomic identifier for H. sapiens. Protein identifiers for human E1 include, but are not limited to, A0AVT1; A6NLB5; A6NN89; O95352; P22314; P41226; Q13564; Q5JRR8; Q5JRR9; and Q9UBT2. “E1” is also used herein to refer to fragments and/or variants thereof that are able to participate in the ubiquitin system as understood in the art and/or described herein.

“Ubiquitin-conjugating enzyme” or “E2” refers to the family of enzyme that, as described above, are involved in small protein conjugating enzyme activity, such as the transfer of Ub/Ubl from E1 and recognition of Ub/Ubl to a target protein. Proteins having E2 activity can be found using the GO term identifiers GO:0019787 and the “Protein Annotation” tab for the particular sequence identifiers. Those skilled in the art would also appreciate that the retrieved proteins can be limited to any individual species. In the case of human proteins, one would use the NCBI taxonomic identifier for H. sapiens. Protein identifiers for human E2 include, but are not limited to, A1L167; A5D8Z3; O00308; O60260; P60604; Q13489; Q5T447; Q8WVN8; Q9HCE7; and Q9Y4X5. “E2” is also used herein to refer to fragments and/or variants thereof that are able to participate in the ubiquitin system as understood in the art and/or described herein.

“Ubiquitin ligase” (or “E3”) are a family of enzymes that, as mentioned above, are involved in ubiquitin-protein ligase activity—recognize and facilitate the transfer of Ub/Ubl from activated E2 to a lysine on a target protein. In addition to acting on a target protein, ubiquitin ligases also may undergo ubiquitylation (“autoubiquitylate”) which may affect the function of the enzyme. Non-limiting examples of E3 include muscle ring-finger protein 1 (“MuRF1”), Hrd1, Parkin, Caspase 8/10-associated RING domain protein1 (“CARP1”); Caspase 8/10-associated RING domain protein2 (“CARP2”), Atrogin1, MDM2, Seven in absentia homolog 2 (“Siah2”), β-transducin repeat containing protein (“β-TrCP”), and Praja1. Proteins having E3 activity can be found using the GO term identifiers GO:0004842 and the “Protein Annotation” tab for the particular sequence identifiers. Those skilled in the art would also appreciate that the retrieved proteins can be limited to any individual species. In the case of human proteins, one would use the NCBI taxonomic identifier for H. sapiens. Protein identifiers for human E3 include, but are not limited to, A1A4G1; A1L491; A2IDB9; A3FG77; A4D1V5; A5D8Z3; A6ND72; A7E2X0; O00308; O14933; O60260; O75426; O94941; P22681; P36406; P38398; P49427; Q06587; Q16763; Q547Q3; and Q5VVX1. “E3” is also used herein to refer to fragments and/or variants thereof that are able to participate in the ubiquitin system as understood in the art and/or described herein.

Ubiquitin ligases are a part of a large family of enzymes—there are believed to be between 500 and 700 different E3s in the human proteome. There are some structural features that identify E3s, which fall into three families. The first family is characterized by the presence of a Homologous to E6AP C-Terminus (“HECT”) domain, which as first identified in the E6-Associated Protein (“E6AP”). The HECT domain is a conserved 350-residue region, which harbors an essential cysteine residue for the ubiquitylation reaction. In HECT E3 catalysis, the E2-bound Ub/Ubl is transferred to the E3 cysteine residue prior to attack of the lysine residue on the target protein.

The second family is characterized by the presence of a Really Interesting New Gene (“RING”) domain, which forms a globular E2-binding domain. The RING domain is a type of zinc finger, containing the Cys₃HisCys₄ motif that coordinate two zinc cations. The RING domain has the consensus sequence C-X₂-C-X_{┌9-39┐}-C-X_{┌1-3┐}-H-X_{┌2-3┐}-C-X₂-C-X_{┌4-48┐}-C-X₂-C (SEQ ID NO:2), in which X is any amino acid, C is a conserved cysteine residue, H is a conserved histidine, and both cysteines and histidines interact with zinc to form the finger conformation. To clarify, in the foregoing consensus sequence “X_{[9-39]}” refers to a string of 9 to 39 amino acids in length, wherein each X may, independently, be any amino acid. Preferably, X is a proteinogenic amino acid. Proteinogenic amino acids are those amino acids that are found in proteins and that are coded for in the standard genetic code. The RING family also includes its derivatives, U-Box and the PHD (Plant Homeo-Domain). RING E3s are scaffolds that dock the charged E2 and the substrate so as to facilitate direct attack of the lysine on the substrate on the E2-linked Ub/Ubl. Some, but not all, RING E3s are multisubunit complexes in which substrate recognition and the catalysis of ubiquitylation are relegated to distinct polypeptides.

The third family is characterized by the presence the N-end rule domains. These ligases get their name from “The N-end rule,” which states that there is a strong relation between the in vivo half-life of a protein and the identity of its N-terminal amino acids. Ligases of this family, such as the human homolog E3α, bind directly to the primary destabilizing N-terminal amino acid. These ligases of have five regions of high similarity, Regions I-V. In region I, the residues Cys-145, Val-146, Gly-173, and Asp-176 are known to be necessary for basic N-terminal substrate binding in yeast and are conserved in the mouse. In regions II and III, residues Asp-318, His-321, and Glu-560 are essential for hydrophobic N-terminal substrate binding in yeast and are also conserved in the mouse. In addition, there is a conserved zinc-finger domain in region I and a conserved RING-H2 domain in region IV.

Different classes of eukaryotic proteins have evolved to contain structural motifs that recognize ubiquitylated proteins. As used herein “ubiquitin-adhering motif-containing protein” refers to the family of proteins that interact with proteins to which Ub/Ubl have been attached. Several “ubiquitin-adhering motifs” have been identified, including: UBA (ubiquitin-associated); UIM (ubiquitin-interacting motif); CUE (coupling of ubiquitin to ER degradation); NZF (Np14 zinc finger); UEV (ubiquitin E2 variant); GLUE (GRAM-like ubiquitin-binding in Eap45); MIU (Motif Interacting with Ubiquitin); and GAT (GAA and Tom1 domain). As used herein “ubiquitin-adhering motif-containing protein” also refers to fragments and/or variants of these proteins that participate in the ubiquitin system as described herein.

The UBA domain, 45 residues, was initially identified in subsets of E2, E3, and USP (ubiquitin-specific protease) superfamilies, and other proteins of functions other than ubiquitylation and deubiquitylation (Hofmann & Bucher, Trends Biochem. Sci. 21: 172-173, 1996). It is usually located at the C-terminus of UBA-containing proteins, although the N-terminal UBA domains have also been characterized. Proteins containing the UBA domains include, for example, HHR23A (the human homolog of yeast RAD23), a protein involved in DNA repair (Watkins et al., Mol. Cell. Biol. 13: 7757-7765, 1993); p62, a protein that mediates diverse cellular functions including control of NF-κB signaling and transcriptional activation (Geetha & Wooten, FEBS Lett. 512: 19-24, 2002); p47, a major adaptor molecule of the cytosolic AAA ATPase p97 (Yuan et al., EMBO J. 23: 1463-1473, 2004); and ubiquilin-2, a protein linking integrin-associated protein and cytoskeleton (Kleijnen et al., Mol. Cell 6: 409-419, 2000). In assays with purified proteins, UBA domains bind monoubiquitin, but have a greater affinity for polyubiquitin chains (Bertolaet et al., Nat. Struct. Biol., 8: 417-422, 2001; Chen et al., EMBO Rep. 2: 933-938, 2001; Wilkinson et al., Nat. Cell Biol., 3:, 939-943, 2001; Funakoshi et al., Proc. Natl Acad. Sci. USA, 99:, 745-750, 2002). The structure of the UBA domain as determined by nuclear magnetic resonance (NMR) is a bundle of three α-helices, and this bundle contains a distinct hydrophobic surface region that is predicted to be the site of interaction with ubiquitin. Dieckmann et al., Nat. Struct. Biol., 5: 1042-1047, 1998; Mueller & Feigon, J. Mol. Biol., 319:, 1243-1255, 2002; Mueller et al., J. Biol. Chem. 279: 11926-11936, 2004.

The UIM domain is a 20 amino acid sequence motif that was identified using iterative database searches with the sequences from the S5a subunit of the proteasome that interact directly with polyubiquitin chains (Hofmann & Falquet, Trends Biochem. Sci., 26: 347-350 2001). UIM's bind to monoubiquitin directly (Polo et al., Nature, 416: 451-455, 2002; Raiborg et al., Nat. Cell Biol., 4:, 394-398, 2002), and are present as tandem pairs or triplets in many proteins. UIM's are found in a number of proteins important in the endocytic pathway (epsins, Eps15 and Hrs), where they are critical for function, and are likely to bind monoubiquitylated partners in the cell (Raiborg et al., Nat. Cell Biol., 4: 394-398, 2002). Other proteins containing UIM's include HRS, S5A, and STAM. Endocytic UIM proteins are themselves monoubiquitylated, and this ubiquitylation event requires the protein's UIM domains (Klapisz et al., J. Biol. Chem., 277: 30746-30753, 2002; Oldham et al., Curr. Biol., 12: 1112-1116, 2002; Polo et al., Nature 416: 451-455, 2002). The UIM domain is made of a single α-helix, centered around a conserved alanine residue, which makes a hydrophobic interaction with ubiquitin. Swanson et al., EMBO J. 22(18): 4597-4606, 2006; see also, Shih et al., Nat. Cell Biol. 4: 389-393, 2002.

The CUE domain was first identified from a yeast two-hybrid screen that could interact with monoubiquitin. Ponting, Biochem. J. 351, 527-535, 2000. The CUE domain is moderately conserved consisting of 40 amino acids and is structurally related to the UBA domain. The domain is composed of a three-helix bundle with a conserved hydrophobic path and both domains interact with ubiquitin analogously. A conserved MFP motif in alpha helix 1 and LL motif in alpha helix 3 interact with the conserved hydrophobic patch of ubiquitin. Kang et al., Cell 113(5), 621-630, 2003. The CUE domain has also been reported to exist as a domain-swapped dimer that makes additional contacts with ubiquitin, and consequently, binds ubiquitin with higher affinity. The CUE domain is found in proteins with diverse functions including degradation of misfolded proteins in the endoplasmic reticulum and protein sorting. Examples of proteins that contain CUE domains include, Vsp9, Cue1, and Tollip. CUE domains recognize both mono and polyubiquitin as well as facilitating intramolecular monoubiquitylation. See also, Davies et al., J. Biol. Chem. 278: 19826-19833, 2003; Shih et al., EMBO J. 22: 1273-1281, 2003.

The NZF domain is a compact zinc-binding module found in many proteins that function in ubiquitin-dependent processes. Present in more than 100 proteins, NZF domains conform to the consensus sequence: X₄-W-X-C-X₂-C-X₃-N-X₆-C-X₂-C-X₅ (SEQ ID NO:3), in which X is any amino acid. Preferably, X is a proteinogenic amino acid. Proteinogenic amino acids are those amino acids that are found in proteins and that are coded for in the standard genetic code. Composed of ˜35 amino acids, the NZF domain forms a compact module composed of four antiparallel β-strands linked by three ordered loops and organized about a rubredoxin-like Zn(Cys)₄ metal-binding site. Alam et al., EMBO J. 23: 1411-1421, 2004. Examples of proteins that contain NZF domains include RanBP2, Vsp36/ESCRT-II, and Np14 zinc finger. See also, Meyer et al., EMBO J. 21: 5645-5652, 2002; Wang et al., J. Biol. Chem. 278: 20225-20234, 2003.

The UEV domain is composed of approximately 145 amino acids and contains a characteristic α/β fold similar to the canonical E2 enzyme, but has an additional N-terminal helix and lacks the two C-terminal helices. Sundquist et al., Mol. Cell 13(6): 783-9, 2004. Found in TSG101Nps23 proteins, the UEV interacts with a ubiquitin molecule and is essential for the trafficking of a number of ubiquitylated cargoes to multivesicular bodies. Furthermore, the UEV domain can bind to Pro-Thr/Ser-Ala-Pro peptide ligands, which is exploited by viruses such as HIV. Thus, the TSG101 UEV domain binds to the PTAP tetrapeptide motif in the viral Gag protein that is involved in viral budding. See also, Garrus et al., Cell 107: 55-65, 2001; Pornillos et al., EMBO J. 21: 2397-2406, 2002.

The MIU domain binds to ubiquitin in a manner almost identical to that of the UIM-Ub interaction, although in the opposite orientation. Similar to UIM domains, a critical Ala residue is required for binding to Ub. MIU-containing proteins have been reported to bind to polyUb chains linked through lysine-48 or lysine-63 of ubiquitin. The identification of MIU domains in proteins such as Myosin VI and Rabex-5 support their role in ubiquitin-dependant vesicular trafficking See, Penengo, Cell 124(6): 1183-1195, 2006.

The GLUE was identified in Eap45, which is the mammalian ortholog of the yeast Vps36, a component of the yeast ESCRT-II complex invoked in vacuolar sorting of ubiquitylated membrane proteins. Slagsvold et al., J. Biol. Chem. 280: 19600-19606, 2005.

The GAT domain is found in GGAs (Golgi-localizing, -adaptin ear domain homology, ADP-ribosylation factor (ARF)-binding proteins), a family of monomeric adaptor proteins involved in membrane trafficking from the trans-Golgi network to endosomes. The C-terminal subdomain of the GAT domain binds ubiquitin. The binding is mediated by interactions between residues on one side of the 3 helix of the GAT domain and those on the so-called Ile-44 surface patch of ubiquitin. See, Collins et al., Dev. Cell. 4(3): 321-332, 2003; Suer, S. et al. (2003) PNAS 100(8): 4451-4456; Shiba, T. et al. (2003) Nat. Struc. Biol. 10(5): 386-392.

Fragments and/or variants of any of the proteins in the ubiquitin system could be used in various embodiments. One skilled in the art would appreciate that such fragments and/or variants may be identified using methods known in the art and/or the methods described herein.

Components of the ubiquitin system may be used in various embodiments in “isolated” form. “Isolated protein” referred to herein means that a subject protein (1) is free of at least some other proteins with which it would normally be found, (2) is essentially free of other proteins from the same source, e.g., from the same species, (3) is expressed by a cell from the same species or a different species, (4) has been separated from at least about 50 percent of polynucleotides, lipids, carbohydrates, or other materials with which it is associated in nature, (5) is not associated (by covalent or noncovalent interaction) with portions of a protein with which the “isolated protein” is associated in nature, (6) is operably associated (by covalent or noncovalent interaction) with a polypeptide with which it is not associated in nature, or (7) does not occur in nature. Such an isolated protein can be encoded by genomic DNA, cDNA, mRNA or other RNA, of synthetic origin, or any combination thereof. Preferably, the isolated protein is substantially free from proteins or polypeptides or other contaminants that are found in its natural environment that would interfere with its use (therapeutic, diagnostic, prophylactic, research or otherwise).

The terms “polypeptide” or “protein” means molecules having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” specifically encompass components of the ubiquitin system, or sequences that have deletions from, additions to, and/or substitutions of one or more amino acid of a component of the ubiquitin system.

The term “protein fragment” refers to a protein that has an amino-terminal deletion, a carboxyl-terminal deletion, and/or an internal deletion. In certain embodiments, fragments are at least 5 to about 500 amino acids long. It will be appreciated that in certain embodiments, fragments are at least 5, 6, 8, 10, 15, 20, 30, 40, 50, 60, 70, 80, 90, 100, 110, 150, 200, 250, 300, 350, 400, 450, 500, 1000, 1500, 2000, 2500, or 3000 amino acids long. Particularly useful peptide fragments include functional domains, including binding domains. In the case of a component of the ubiquitin system, useful fragments include but are not limited to a HECT, RING, N-end Rule Domain, and ubiquitin-adhering motif.

Preferred methods to determine identity are designed to give the largest match between the sequences tested. Methods to determine identity are described in publicly available computer programs. Preferred computer program methods to determine identity between two sequences include, but are not limited to, the GCG program package, including GAP (Devereux et al., 1984, Nucl. Acid. Res., 12:387; Genetics Computer Group, University of Wisconsin, Madison, Wis.), BLASTP, BLASTN, and FASTA (Altschul et al., 1990, J. Mol. Biol., 215:403-410). The BLASTX program is publicly available from the National Center for Biotechnology Information (NCBI) and other sources (BLAST Manual, Altschul et al. NCB/NLM/NIH Bethesda, Md. 20894; Altschul et al., 1990, supra). The well-known Smith Waterman algorithm may also be used to determine identity.

Certain alignment schemes for aligning two amino acid sequences may result in matching of only a short region of the two sequences, and this small aligned region may have very high sequence identity even though there is no significant relationship between the two full-length sequences. Accordingly, in certain embodiments, the selected alignment method (GAP program) will result in an alignment that spans at least 50 contiguous amino acids of the target peptide.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span,” as determined by the algorithm). In certain embodiments, a gap opening penalty (which is calculated as three-times the average diagonal; where the “average diagonal” is the average of the diagonal of the comparison matrix being used; the “diagonal” is the score or number assigned to each perfect amino acid match by the particular comparison matrix) and a gap extension penalty (which is usually one-tenth of the gap opening penalty), as well as a comparison matrix such as PAM250 or BLOSUM 62 are used in conjunction with the algorithm. In certain embodiments, a standard comparison matrix (see Dayhoff et al., 1978, Atlas of Protein Sequence and Structure, 5:345-352 for the PAM 250 comparison matrix; Henikoff et al., 1992, Proc. Natl. Acad. Sci USA, 89:10915-10919 for the BLOSUM 62 comparison matrix) is also used by the algorithm.

The term “homology” refers to the degree of similarity between protein or nucleic acid sequences. Homology information is useful for the understanding the genetic relatedness of certain protein or nucleic acid species. Homology can be determined by aligning and comparing sequences. Typically, to determine amino acid homology, a protein sequence is compared to a database of known protein sequences. Homologous sequences share common functional identities somewhere along their sequences. A high degree of similarity or identity is usually indicative of homology, although a low degree of similarity or identity does not necessarily indicate lack of homology.

Several approaches can be used to compare amino acids from one sequence to amino acids of another sequence to determine homology. Generally, the approaches fall into two categories: (1) comparison of physical characteristics such as polarity, charge, and Van der Waals volume, to generate a similarity matrix; and (2) comparison of likely substitution of an amino acid in a sequence by any other amino acid, which is based on observation of many protein sequences from known homologous proteins and to generate a Point Accepted Mutation Matrix (PAM).

The percentage of identity may also be calculated by using the program needle (EMBOSS package) or stretcher (EMBOSS package) or the program align X, as a module of the vector NTI suite 9.0.0 software package, using the default parameters (for example, GAP penalty 5, GAP opening penalty 15, GAP extension penalty 6.6).

As used herein, the twenty conventional amino acids and their abbreviations follow conventional usage. See IMMUNOLOGY—A SYNTHESIS, 2nd Edition, (E. S. Golub and D. R. Gren, Eds.), Sinauer Associates: Sunderland, Mass., 1991, incorporated herein by reference for any purpose. Stereoisomers (e.g., D-amino acids) of the twenty conventional amino acids; unnatural amino acids such as α, α-disubstituted amino acids, N-alkyl amino acids, lactic acid, and other unconventional amino acids may also be suitable components for polypeptides in various embodiments of the invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

Naturally occurring residues may be divided into classes based on common side chain properties:

1) hydrophobic: norleucine (Nor), Met, Ala, Val, Leu, Ile, Phe, Trp, Tyr, Pro;

2) polar hydrophilic: Arg, Asn, Asp, Gln, Glu, His, Lys, Ser, Thr;

3) aliphatic: Ala, Gly, Ile, Leu, Val, Pro;

4) aliphatic hydrophobic: Ala, Ile, Leu, Val, Pro;

5) neutral hydrophilic: Cys, Ser, Thr, Asn, Gln;

6) acidic: Asp, Glu;

7) basic: His, Lys, Arg;

8) residues that influence chain orientation: Gly, Pro;

9) aromatic: His, Trp, Tyr, Phe; and

10) aromatic hydrophobic: Phe, Trp, Tyr.

Conservative amino acid substitutions may involve exchange of a member of one of these classes with another member of the same class. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties.

Non-conservative substitutions may involve the exchange of a member of one of these classes for a member from another class. Such substituted residues may be introduced into regions of the human antibody that are homologous with non-human antibodies, or into the non-homologous regions of the molecule.

In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those that are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as disclosed herein. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5.+−.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those that are within ±1 are included, and in certain embodiments, those within ±0.5 are included.

Exemplary amino acid substitutions are set forth in the Table below:

Amino Acid Substitutions

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, | Ala, Leu |
| | Phe, Norleucine | |
| Leu | Norleucine, Ile, Val, Met, | Ile |
| | Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, | Leu |
| | Norleucine | |

A skilled artisan will be able to determine suitable variants of the protein as set forth herein using well-known techniques. In certain embodiments, one skilled in the art may identify suitable areas of the molecule that may be changed without destroying activity by targeting regions not believed to be important for activity. In other embodiments, the skilled artisan can identify residues and portions of the molecules that are conserved among similar proteins. In further embodiments, even areas that may be important for biological activity or for structure may be subject to conservative amino acid substitutions without destroying the biological activity or without adversely affecting the protein structure.

Additionally, one skilled in the art can review structure-function studies, such as the ubiquitin ligase assay described herein, to identify residues in similar proteins that are important for activity or structure. In view of such a comparison, the skilled artisan can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins. In certain embodiments, one skilled in the art may choose to not make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays described herein. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult, 1996, Curr. Op. in Biotech. 7:422-427; Chou et al., 1974, Biochemistry 13:222-245; Chou et al., 1974, Biochemistry 113:211-222; Chou et al., 1978, Adv. Enzymol. Relat. Areas Mol. Biol. 47:45-148; Chou et al., 1979, Ann. Rev. Biochem. 47:251-276; and Chou et al., 1979, Biophys. J. 26:367-384. Moreover, computer programs are currently available to assist with predicting secondary structure. One method of predicting secondary structure is based upon homology modeling. For example, two polypeptides or proteins that have a sequence identity of greater than 30%, or similarity greater than 40% often have similar structural topologies. The recent growth of the protein structural database (PDB) has provided enhanced predictability of secondary structure, including the potential number of folds within a polypeptide's or protein's structure. See Holm et al., 1999, Nucl. Acid. Res. 27:244-247. It has been suggested (Brenner et al., 1997, Curr. Op. Struct. Biol. 7:369-376) that there are a limited number of folds in a given polypeptide or protein and that once a critical number of structures have been resolved, structural prediction will become dramatically more accurate.

### Effect of Ubiquitylation

The ubiquitin system has been implicated in a number of biological processes, such as antigen processing, apoptosis, cell cycle and division, DNA repair, differentiation and development, endocytosis and exocytosis, gene silencing, immune responses and inflammation, muscular degeneration, neural degeneration, neural development, organellar development, protein degradation, signal transduction from cell surface receptors and channels, stress responses, and transcription.

The effect of ubiquitylation varies depending, for instance, upon the amount of Ub/Ubl attached (e.g., mono-versus polyubiquitylation), the location of the Ub/Ubl attachment (e.g., multiple monoubiquitylation), and the identity of the substrate (e.g., transcription factor, receptor, or structural protein). In some instances UB/Ubl mark a substrate for degradation. K48-linked polyubiquitin chains are involved in marking proteins as substrates for the 26S proteasome. In contrast, monoubiquitylation and K63-linked chains generally serve as signals for non-degradative events such as endocytosis, vesicular trafficking, cell-cycle control, stress response, DNA repair signaling, transcription, and gene silencing. Ultimately, ubiquitylation serves as a signaling event that could lead to a multitude of occurrences dependent on the chain-linkage.

“Aberrant” ubiquitylation refers to a state or condition in which the attachment or removal of Ub/Ubls differs from the norm. Aberrant ubiquitylation may manifest in a number of undesirable scenarios, such as cancer, immune suppression, muscular degeneration and wasting, neurodegeneration. The ubiquitin system has been implicated in certain histological abnormalities, including abnormal accumulations of protein inside the cell, or inclusion bodies. Examples of conditions displaying such abnormal inclusions include neurofibrillary tangles in Alzheimer's disease, Lewy bodies in Parkinson's disease; Pick bodies in Pick's disease; inclusions in motor neuron disease, Mallory' bodies in alcoholic liver disease, and Rosenthal fibres in Alexander disease. The ubiquitin system has also been implicated in certain genetic disorders, which include a role for a UBE3A gene disruption in Angelman syndrome, VHL tumor suppressor (VHL) gene disruption in Von Hippel-Lindau syndrome, epithelial Na+ channel (ENaC) gene disruption in Liddle's Syndrome, and various genes that are thought to be disrupted in Fanconi anemia. Using methods known the art and/or described herein, a skilled artisan would be able to identify aberrant ubiquitin activity that occurs under certain conditions, diseases, and/or syndromes.

### Modulators of Ubiquitylation

The term “modulator” means a compound which can increase (an “activator”) or decrease (an “inhibitor”) ubiquitin ligase activity. The terms “candidate”, “candidate agent”, “candidate modulator,” and “candidate ubiquitin ligase activity modulator” refer to any molecule, e.g. proteins (which herein includes proteins, polypeptides, and peptides), small organic or inorganic molecules, polysaccharides, polynucleotides, etc. which are to be tested for ubiquitin ligase activity modulator activity. Candidate agents encompass numerous chemical classes. In a preferred embodiment, the candidate agents are organic molecules, particularly small organic molecules, comprising functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more chemical functional groups.

Candidate modulators are obtained from a wide variety of sources, as will be appreciated by those in the art, including libraries of synthetic or natural compounds. As will be appreciated by those in the art, embodiments of the invention provide a rapid and easy method for screening any library of candidate modulators, including the wide variety of known combinatorial chemistry-type libraries.

In certain aspects, candidate modulators are synthetic compounds. A number of techniques are available for the random and directed synthesis of a wide variety of organic compounds and biomolecules, including expression of randomized oligonucleotides. Alternatively, other aspects use libraries of natural compounds in the form of bacterial, fungal, plant and animal extracts that are available or readily produced. Moreover, natural or synthetically produced libraries and compounds are readily modified through conventional chemical, physical and biochemical means. Known pharmacological agents may be subjected to directed or random chemical modifications, including enzymatic modifications, to produce structural analogs.

Where the candidate modulators are proteins, they may be naturally occurring proteins or fragments of naturally occurring proteins. Thus, for example, cellular extracts containing proteins, or random or directed digests of proteinaceous cellular extracts, may be tested. In this way libraries of prokaryotic and eukaryotic proteins may be made for screening against any number of ubiquitin ligase compositions. Particularly preferred in this embodiment are libraries of bacterial, fungal, viral, and mammalian proteins, with the latter being preferred, and human proteins being especially preferred.

In other aspects, the candidate modulators are peptides ranging in size from about 2 to about 50 amino acids, with from about 5 to about 30 amino acids being preferred, and from about 8 to about 20 being particularly preferred. The peptides may be digests of naturally occurring proteins as is outlined above, random peptides, or “biased” random peptides. The term “randomized” is intended to mean that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

Where the embodiment uses a library, the library should provide a sufficiently structurally diverse population of randomized agents to effect a probabilistically sufficient range of diversity to allow interaction with a particular ubiquitin ligase enzyme. Accordingly, an interaction library must be large enough so that at least one of its members will have a structure that interacts with a ubiquitin ligase enzyme. Those skilled in the art would understand how to best construct a sufficiently large and diverse library.

Further embodiments relate to a fully randomized library, with no sequence preferences or constants at any position. In other aspects, the library is biased, wherein some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

In some aspects, the candidate modulators are nucleic acids. With reference to candidate modulators, “nucleic acid” or “oligonucleotide” used herein means at least two nucleotides covalently linked together. Embodiments composed of nucleic acids will generally contain phosphodiester bonds, although in some cases, as outlined below, nucleic acid analogs are included that may have alternate backbones, comprising, for example, phosphoramide, phosphorothioate, phosphorodithioate, O-methylphophoroamidite linkages, and peptide nucleic acid backbones and linkages. Other analog nucleic acids include those with positive backbones; non-ionic backbones, and non-ribose backbones. Nucleic acids containing one or more carbocyclic sugars are also included within the definition of nucleic acids. These modifications of the ribose-phosphate backbone may be done to facilitate the addition of additional moieties such as labels, or to increase the stability and half-life of such molecules in physiological environments. As will be appreciated by those in the art, all of these nucleic acid analogs may find use in various inventive embodiments. In addition, mixtures of naturally occurring nucleic acids and analogs can be made. Alternatively, mixtures of different nucleic acid analogs, and mixtures of naturally occurring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids.

Further embodiments include candidate modulators that are organic moieties, which can be synthesized from a series of substrates that can be chemically modified. “Chemically modified” includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested in various embodiments.

As will be appreciated by those in the art, it is possible to screen more than one type of candidate modulator at a time. Thus, the library of candidate modulators used may include only one type of agent (i.e. peptides), or multiple types (peptides and organic agents). The assay of several candidates at one time is further discussed below.

### Ubiquitylation Ligase Assays

Certain embodiments provide methods of combining components of the ubiquitylation system. By “combining” is meant the addition of the various components into a receptacle under conditions whereby ubiquitin ligase activity may take place. In a preferred embodiment, the receptacle is a well of a 96-well plate or other commercially available multiwell plate. In an alternate preferred embodiment, the receptacle is the reaction vessel of a FACS machine. Other receptacles include, but are not limited to 384 well plates and 1536 well plates. Still other suitable receptacles will be apparent to the skilled artisan.

Certain embodiments relate to having a “solid support” either as part of or added separately to the receptacle in which the ubiquitin ligase activity may take place. A ubiquitin- adhering motif-containing protein (or fragments thereof) may be bound to the solid support. The solid support may be any number of materials, including inorganic polymers, especially glass, silica, metal oxides; or organic polymers, especially cellulose, or optionally substituted polystyrene. These materials may be used as microbeads or agglomerated microfibers. Still other suitable materials will be apparent to the skilled artisan.

The addition of the components may be sequential or in a predetermined order or grouping, as long as the conditions amenable to ubiquitin ligase activity are obtained. Such conditions are well known in the art, and further guidance is provided below.

The components of the present compositions may be combined in varying amounts. In a preferred embodiment, Ub/Ubl is combined at a final concentration of from about 1 to 1000 ng per reaction solution ranging from about 4 to 200 μl, most preferable at about 500 ng per 30 μl reaction solution.

In a preferred embodiment, E1 is combined at a final concentration of about 1 nM, 2 nM, 3 nM, 4 nM, 5 nM, 6 nM, 7 nM, 8 nM, 9 nM, 10 nM, 20 nM, 30 nM, 50 nM, 100 nM, 120 nM, 150 nM, 175 nM, 200 nM, 250 nM, 300 nM, 350 nM, 400 nM, 450 nM, 500 nM, 550 mM, 600 nM, 650 nM, 700 nM, 750 nM, 800 nM, 850 nM, 900 nM, 1 μM, 10 μM, or 1 mM.

In a preferred embodiment, E2 is combined at a final concentration of about 1 nM, 2 nM, 3 nM, 4 nM, 5 nM, 6 nM, 7 nM, 8 nM, 9 nM, 10 nM, 20 nM, 30 nM, 50 nM, 100 nM, 120 nM, 150 nM, 175 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 1 μM, 10 μM, or 1 mM.

In a preferred embodiment, E3 is combined at a final concentration of about 1 nM, 2 nM, 3 nM, 4 nM, 5 nM, 6 nM, 7 nM, 8 nM, 9 nM, 10 nM, 20 nM, 30 nM, 50 nM, 100 nM, 120 nM, 150 nM, 175 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 1 μM, 10 μM, or 1 mM.

The components of the ubiquitin system are combined under reaction conditions that favor ubiquitin ligase activity. Generally, this will be physiological conditions. Incubations may be performed at any temperature which facilitates optimal activity, typically between about 4 and 40° C. Incubation periods are selected for optimum activity, but may also be optimized to facilitate rapid high through put screening. Incubations may be performed for times that facilitate optimal activity, such as about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 hours. Typically between about 0.1 and about 2.0 hours will be sufficient.

A variety of other reagents may be included in the assay. These include reagents like salts, solvents, buffers, neutral proteins, e.g. albumin, detergents, etc. which may be used to facilitate optimal ubiquitylation enzyme activity and/or reduce non-specific or background interactions. Also reagents that otherwise improve the efficiency of the assay, such as protease inhibitors, nuclease inhibitors, anti-microbial agents, etc., may be used. The compositions will also preferably include adenosine triphosphate (ATP).

The mixture of components may be added in any order that promotes ubiquitin ligase activity or optimizes identification of candidate modulator effects. In a preferred embodiment, ubiquitin is provided in a reaction buffer solution, followed by addition of the ubiquitylation enzymes. In an alternate preferred embodiment, ubiquitin is provided in a reaction buffer solution; a candidate modulator is then added, followed by addition of the ubiquitylation enzymes.

Once combined, preferred embodiments comprise measuring the amount of ubiquitin bound to E3 or a substrate. As will be understood by one of ordinary skill in the art, the mode of measuring will depend on the specific tag attached to a component of the assay, most preferably Ub/Ubl. As will also be apparent to the skilled artisan, the amount of ubiquitin bound will encompass not only the particular ubiquitin protein bound directly to the ubiquitylation enzyme, but also the ubiquitin proteins bound to the former in a polyubiquitin chain.

In a preferred embodiment, the tag attached to the ubiquitin is a fluorescent label. In a preferred embodiment, the tag attached to ubiquitin is an enzyme label or a binding pair member which is indirectly labeled with an enzyme label. In this latter preferred embodiment, the enzyme label substrate produces a fluorescent reaction product. In these preferred embodiments, the amount of ubiquitin bound is measured by luminescence. Equipment for such measurement is commercially available and easily used by one of ordinary skill in the art to make such a measurement.

Other modes of measuring bound ubiquitin are well known in the art and easily identified by the skilled artisan for each of the labels described herein. For instance, radioisotope labeling may be measured by scintillation counting, or by densitometry after exposure to a photographic emulsion, or by using a device such as a Phosphorimager. Likewise, densitometry may be used to measure bound ubiquitin following a reaction with an enzyme label substrate that produces an opaque product when an enzyme label is used.

In preferred embodiments, the ubiquitin-adhering motif-containing protein (or fragment thereof) is bound to a solid support. This may be done directly or by using a linker or tag, such as His, GST, and the like, that is attached to the ubiquitin-adhering motif-containing protein (or fragment thereof), wherein the adapter is a surface substrate binding molecule.

Other aspects relate to ubiquitin-adhering motif-containing proteins (or fragments thereof), bound, directly or via a substrate binding element, to a bead. Following ligation, the beads may be separated from the unbound ubiquitin and the bound ubiquitin measured. In a preferred embodiment, ubiquitin-adhering motif-containing proteins (or fragments thereof) are bound to beads and the composition used includes tag-ubiquitin wherein tag is a fluorescent label. In this embodiment, the beads with bound ubiquitin may be separated using a fluorescence-activated cell sorting (FACS) machine. The amount of bound ubiquitin can then be measured.

In a preferred embodiment, multiple assays are performed simultaneously in a high throughput screening system. In this embodiment, multiple assays may be performed in multiple receptacles, such as the wells of a 96 well plate or other multi-well plate. As will be appreciated by one of skill in the art, such a system may be applied to the assay of multiple candidate modulators and/or multiple combinations of the ubiquitin system components. In a preferred embodiment, a high-throughput screening system may be used for determining the ubiquitin ligase activity of different E3s—candidate modulator pairings and/or different target protein-candidate modulator combinations. Other features relate to a high throughput screening system for simultaneously testing the effect of individual candidate modulators.

It is understood by the skilled artisan that the steps of the assays provided herein can vary in order. It is also understood, however, that while various options (of compounds, properties selected or order of steps) are provided herein, the options are also each provided individually, and can each be individually segregated from the other options provided herein. Moreover, steps which are obvious and known in the art that will increase the sensitivity of the assay are intended to be within the scope of this invention. For example, there may be additionally washing steps, blocking steps, etc.

### Methods of Detecting Ubiquitylation

In further embodiments, one or more components of the ubiquitin ligase assay comprise a tag. By “tag” is meant an attached molecule or molecules useful for the identification or isolation of the attached component. Components having a tag are referred to as “tag-X”, wherein X is the component. For example, a ubiquitin comprising a tag is referred to herein as “tag-ubiquitin”. Preferably, the tag is covalently bound to the attached component. When more than one component of a combination has a tag, the tags will be numbered for identification, for example “tag1-ubiquitin”. Preferred tags include, but are not limited to, a label, a partner of a binding pair, and a surface substrate binding molecule. As will be evident to the skilled artisan, many molecules may find use as more than one type of tag, depending upon how the tag is used.

In a preferred embodiment, ubiquitin is in the form of tag-ubiquitin. In another preferred embodiment, E3 has a tag, which complex is referred to herein as “tag-E3.” Preferably, the tag is attached to only one component of the E3. Preferred E3 tags include, but are not limited to, labels, partners of binding pairs and substrate binding elements.

By “label” is meant a molecule that can be directly (i.e., a primary label) or indirectly (i.e., a secondary label) detected; for example a label can be visualized and/or measured or otherwise identified so that its presence or absence can be known. As will be appreciated by those in the art, the manner in which this is done will depend on the label. Preferred labels include, but are not limited to, fluorescent labels, label enzymes, and radioisotopes.

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malachite green, stilbene, Lucifer Yellow, Cascade Blue™, and Texas Red. Suitable optical dyes are described in the 1996 Molecular Probes Handbook by Richard P. Haugland, hereby expressly incorporated by reference. Suitable fluorescent labels also include, but are not limited to, green fluorescent protein (GFP), blue fluorescent protein (BFP), enhanced yellow fluorescent protein (EYFP), luciferase, β-galactosidase, and Renilla.

By “label enzyme” is meant an enzyme which may be reacted in the presence of a label enzyme substrate which produces a detectable product. Suitable label enzymes include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol, have been developed that produce fluorescent reaction products. Methods for identifying label enzymes with label enzyme substrates are well known in the art and many commercial kits are available.

By “radioisotope” is meant any radioactive molecule. Suitable radioisotopes include, but are not limited to ¹⁴C, ³H, ³²P, ³³P, ³⁵S, ¹²⁵I, and ¹³¹I. The use of radioisotopes as labels is well known in the art.

In addition, labels may be indirectly detected, that is, the tag is a partner of a binding pair. By “partner of a binding pair” is meant one of a first and a second moiety, wherein said first and said second moiety have a specific binding affinity for each other. Suitable binding pairs include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as FLAG; the KT3 epitope peptide; tubilin epitope peptide; and the T7 gene 10 protein peptide tag, and the antibodies each thereto. Generally, in a preferred embodiment, the smaller of the binding pair partners serves as the tag, as steric considerations in ubiquitin ligation may be important. As will be appreciated by those in the art, binding pair partners may be used in applications other than for labeling, as is further described below.

As will be appreciated by those in the art, a partner of one binding pair may also be a partner of another binding pair. For example, an antigen (first moiety) may bind to a first antibody (second moiety) which may, in turn, be an antigen to a second antibody (third moiety). It will be further appreciated that such a circumstance allows indirect binding of a first moiety and a third moiety via an intermediary second moiety that is a binding pair partner to each.

As will be appreciated by those in the art, a partner of a binding pair may comprise a label, as described above. It will further be appreciated that this allows for a tag to be indirectly labeled upon the binding of a binding partner comprising a label. Attaching a label to a tag which is a partner of a binding pair, as just described, is referred to herein as “indirect labeling”.

As will be appreciated by those in the art, tag-components can be made in various ways, depending largely upon the form of the tag. Components and tags are preferably attached by a covalent bond. The production of tag-polypeptides by recombinant means when the tag is also a polypeptide is described below.

Biotinylation of target molecules and substrates is well known, for example, a large number of biotinylation agents are known, including amine-reactive and thiol-reactive agents, for the biotinylation of proteins, nucleic acids, carbohydrates, carboxylic acids. A biotinylated substrate can be attached to a biotinylated component via avidin or streptavidin. Similarly, a large number of haptenylation reagents are also known.

Methods for labeling of proteins with radioisotopes are known in the art.

Production of proteins having His-tags by recombinant means is well known, and kits for producing such proteins are commercially available.

The functionalization of labels with chemically reactive groups such as thiols, amines, carboxyls, etc. is generally known in the art. In a preferred embodiment, the tag is functionalized to facilitate covalent attachment.

The covalent attachment of the tag may be either direct or via a linker. In one embodiment, the linker is a relatively short coupling moiety, that is used to attach the molecules. A coupling moiety may be synthesized directly onto a component of the ubiquitin ligase assay, ubiquitin for example, and contains at least one functional group to facilitate attachment of the tag. Alternatively, the coupling moiety may have at least two functional groups, which are used to attach a functionalized component to a functionalized tag, for example. In an additional embodiment, the linker is a polymer. In this embodiment, covalent attachment is accomplished either directly, or through the use of coupling moieties from the component or tag to the polymer. In a preferred embodiment, the covalent attachment is direct, that is, no linker is used. In this embodiment, the component preferably contains a functional group such as a carboxylic acid which is used for direct attachment to the functionalized tag. It should be understood that the component and tag may be attached in a variety of ways, including those listed above. What is important is that manner of attachment does not significantly alter the functionality of the component. For example, in tag-ubiquitin, the tag should be attached in such a manner as to allow the ubiquitin to be covalently bound to other ubiquitin to form polyubiquitin chains. As will be appreciated by those in the art, the above description of covalent attachment of a label and ubiquitin applies equally to the attachment of virtually any two molecules of the present disclosure.

In certain embodiments, the tag is functionalized to facilitate covalent attachment, as is generally outlined above. Thus, a wide variety of tags are commercially available which contain functional groups, including, but not limited to, isothiocyanate groups, amino groups, haloacetyl groups, maleimides, succinimidyl esters, and sulfonyl halides, all of which may be used to covalently attach the tag to a second molecule, as is described herein. The choice of the functional group of the tag will depend on the site of attachment to either a linker, as outlined above or a component of the ubiquitin ligase assay. Thus, for example, for direct linkage to a carboxylic acid group of a ubiquitin, amino modified or hydrazine modified tags will be used for coupling via carbodiimide chemistry, for example using 1-ethyl-3-(3-dimethylaminopropyl)-carbodiimide (EDAC) as is known in the art. In one embodiment, the carbodiimide is first attached to the tag, such as is commercially available for many of the tags described herein.

Further embodiments involve using cloned and expressed components (including fragments) of the ubiquitin system, including target proteins. The processes involved in cloning and expression, such as polymerase chain reactions, expression vectors, cellular transfection and transformation, are well known in the art.

Components of the ubiquitin system may also be made as a fusion protein, using techniques well known in the art. Thus, for example, the protein may be made as a fusion protein to increase expression, or for other reasons. For example, when the protein is a peptide, the nucleic acid encoding the peptide may be linked to other nucleic acid for expression purposes. Similarly, components of the ubiquitin system may be linked to protein labels, such as green fluorescent protein (GFP), red fluorescent protein (RFP), blue fluorescent protein (BFP), yellow fluorescent protein (YFP), etc.

In addition the other methods described above, one skilled in the art would recognize that other detection methods would also be suitable in various embodiments, such as fluorescence polarization, fluorescence resonance transfer, or chromogenicity.

The various ubiquitin systems components may be purified or isolated after expression. Proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the ubiquitin protein may be purified using a standard anti-ubiquitin antibody column. Ultrafiltration and diafiltration techniques, in conjunction with protein concentration, are also useful. For general guidance in suitable purification techniques, see Scopes, R., Protein Purification, Springer-Verlag, N.Y. (1982). The degree of purification necessary will vary depending on the use of the protein. In some instances no purification will be necessary.

Various embodiments provide methods for identifying modulators of Ub and Ubl ligases. Certain aspects of the invention involve combining Ub/Ubl, enzymes of the ubiquitylation cascade, including the ligase, and a ubiquitin-adhering motif-contain protein (or fragments thereof) and measuring the amount of ubiquitin bound to the ubiquitin ligase. This aspect of the invention may be useful in identifying components of a particular Ub/Ubl system and defining the basal level of auto-ubiquitylation of a ligase in the absence of any candidate modulators if the ligase activity. Further aspects of the invention include the addition of a candidate ligase modulator and comparing the level of ubiquitylation in the presence of a modulator with the level of ubiquitylation in the absence of a modulator.

Other embodiments relate to combining Ub/Ubl, enzymes of the ubiquitylation cascade, including the ligase, a ubiquitin-adhering motif-contain protein (or fragments thereof), and a target protein; and measuring the amount of ubiquitin bound to the target protein. This aspect of the invention may be useful in identifying components of a particular Ub/Ubl system and defining the basal level of a ubiquitylation of a target protein in the absence of any candidate modulators if the ligase activity. Further aspects of the invention include the addition of a candidate ligase modulator and comparing the level of ubiquitylation in the presence of a modulator with the level of ubiquitylation in the absence of a modulator.

Certain embodiments relate to tagging components of the assay. In a preferred embodiment Ub/Ubl contains a tag. Preferably the tag is a label, a partner of a binding pair, or a substrate binding molecule. More preferably, the tag is a fluorescent label or a binding pair partner. In a preferred embodiment, the tag is a binding pair partner and the ubiquitin is labeled by indirect labeling. In the indirect labeling embodiment, preferably the label is a fluorescent label or a label enzyme. In an embodiment comprising a label enzyme, preferably the substrate for that enzyme produces a luminescent product. In a preferred embodiment, the label enzyme substrate is luminol.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. Thus, various embodiments also relate to ubiquitin ligase assays that use other ubiquitin-adhering-containing motifs and ligases or putative ligases that are not described in the Examples below. All references cited herein are expressly incorporated by reference in their entirety.

### EXAMPLES

### Example 1

### 6× His-SUMO-UBA2 and 6× His-SUMO^{G2C}-UBA1 Expression and Purification

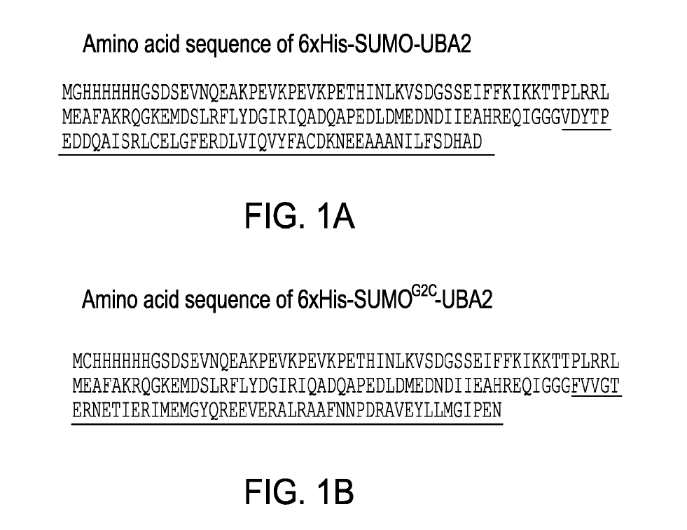

An amino terminal 6× His-SUMO-UBA2 was constructed as follows. A PCR product was generated using primers 5′-GATCGGTCTCAAGGTGTTGACTATACCCCCGAAGA-3′(SEQ ID NO:4) and 5′-GATCGGATCCTCAGTCGGCATGATCGCTGA-3′ (SEQ ID NO:5) using yeast RAD23 gene (residues 351-398 of yRad23 protein) as template from genomic DNA (S. cerevisiae). The PCR fragment was digested with BsaI and BamHI, and ligated into p6× His-SUMO plasmid (Life Sensors). The plasmid was sequenced to confirm the presence of the correct sequence (Genewiz). FIG. 1A shows the amino acid sequence of 6× His-SUMO-UBA2 with the UBA2 domain underlined.

An amino terminal 6× His-SUMO^{G2C}-UBA1 was constructed as follows. A PCR product was generated using primers 5′-GATCCGTCTCAAGGTGGATTCGTGGTGGG AACCGAG-3′(SEQ ID NO:6) and 5′-GATCCGTCTCAGATCCTAATTTTCTGGAATACCCATCAG-3′ (SEQ ID NO:7) using yeast RAD23 gene as template. The PCR fragment was digested with BsaI and then ligated into p6× His-SUMO plasmid (Life Sensors). The glycine at position 2 of 6× His-SUMO was changed to cysteine by site directed mutagenesis using primers 5′-GAAGGAGATATACCATGTGTCATCACCATCATCATCACG-3′ (SEQ ID NO:8) and 5′-CGTGATGATGATGGTGATGACACATGGTATATCTCCTTC-3′ (SEQ ID NO:9). The plasmid was sequenced to confirm the presence of correct sequence. FIG. 1B shows the amino acid sequence of 6× His-SUMO^{G2C}-UBA1 with the UBA1 domain underlined.

To express these fusion proteins, a single colony of the E. coli strain BL21 containing the relevant plasmid was inoculated into 50 ml of Luria-Bertani (LB) media containing 100 μg/mlkanamycin. The cells were grown at 37° C. overnight with shaking at 250 rpm. The next morning 10 ml of the overnight culture was transferred into 1 L of fresh medium to permit exponential growth. When the OD₆₀₀ value reached ˜0.5-0.6, protein expression was induced by addition of 0.1 mM IPTG (isopropropyl-β-D-thiogalactopyranoside), followed by incubation at 20° C. overnight (˜15 hours).

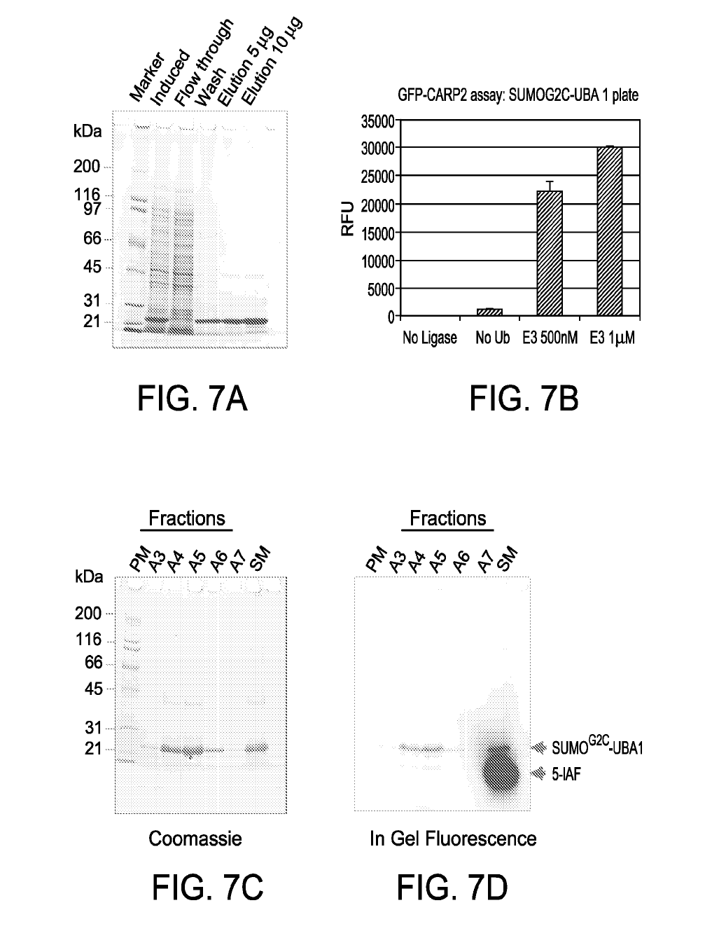

E. coli cells were harvested from 1 L LB medium by centrifugation (4,000×g for 20 minutes at 4° C.), the cell pellets were suspended in 25 ml of lysis buffer (20 mM Tris-HCl, pH8.0, 300 mM NaCl, 20 mM imidazole, 1 mM Phenylmethylsulfonyl fluoride).The cells were lysed by sonication. The lysates were centrifuged at 11,400 rpm for 20 minutes at 4° C., and the supernatant (soluble protein fractions) were collected. The supernatant was then passed through 2 ml Ni-NTA Sepharose (Invitrogen) packed into a column. The column was washed with 10 column volumes of washing buffer (20 mM Tris-HCl, pH8.0, 300 mM NaCl, 20 mM imidazole) and eluted with 5 column volumes of elution buffer (20 mM potassium phosphate, pH8.0, 500 mM NaCl, 500 mM imidazole). Aliquots from the flow-through wash and elution were loaded onto 4-20% SDS-PAGE gradient gels and stained with coomassie brilliant blue. FIG. 7A shows that UBA1 can be expressed and purified to near homogeneity.

### Example 2

### Affigel Coupling of 6× His-SUMO-UBA2

6× His-SUMO-UBA2 was dialyzed with 50 mM MOPS overnight at 4° C. (dialysis tubing MWCO 12-14,000). 2 ml of 6× His-SUMO-UBA2 was transferred into 4 ml of Affige1-15 and agitated on a shaker for 4 hours at 4° C. 1M ethanolamine HCl (pH 8.0) was added (0.4 ml) to block any active esters on the Affigel and incubated for 1 hour at room temperature on a shaker. This was washed with water, followed by 2M NaCl, and stored in 20% ethanol at 4° C.

### Example 3

### E3 Ligase Assay

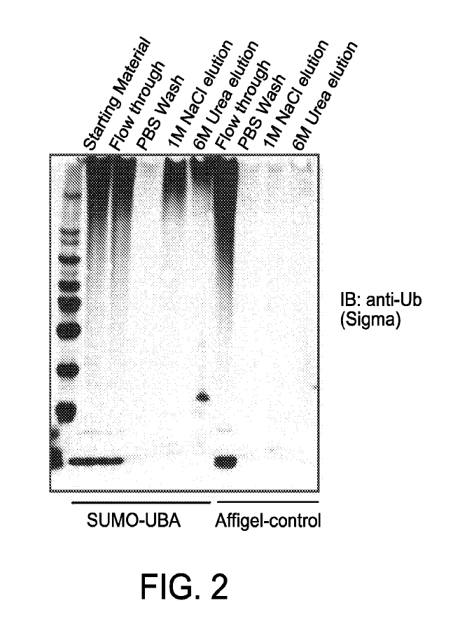

E3 ligase assays consisted of 500 nM 6× His-SUMO-CARP2, 50 nM E1 (BIOMOL), 150 nM UbcH5c (E2) (BIOMOL), 500 ng Ubiquitin, 2 mM ATP, 50 mM Tris-HCl pH8.0, 5 mM MgCl₂, 2 mM DTT. In most cases ubiquitin may contain a 6× His tag. A PCR product encoding CARP2 was generated using the primers 5′-GATCCGTCTCAAGGTATGTGGGCAACCTGCTGCAA-3′(SEQ ID NO:10) and 5′-GATCGGATCCTCAGGACCGGAAGACATGCA-3′ (SEQ ID NO:11) and human CARP2 cDNA as the template. The PCR fragment was digested with BsmBI and BamHI, and then ligated into p6× His-SUMO plasmid (LifeSensors) to generate 6× His-SUMO-CARP2. The plasmid was sequenced to confirm the presence of the correct sequence. 30 μl of the reaction mixture was incubated for 90 minutes at 37° C. and transferred to a 3 ml column containing 50 μl bed volume of Affigel coupled to 6× His-SUMO-UBA2. The column was washed with 5 column volumes of washing buffer (1× phosphate buffer saline (PBS)) and eluted with 5 column volumes of elution buffer as well as denaturing buffer (50 mM Tris-HCl, pH8.0, 1M NaCl and Tris-HCl, pH8.0, 1M NaCl, 6M UREA, respectively). Aliquots from the flow-through wash, elution, and denaturing buffer were loaded onto 4-20% SDS-PAGE gradient gels and probed with an anti-ubiquitin antibody (Sigma) and an anti-rabbit HRP-conjugated antibody (Jackson ImmunoResearch) by Western Blot (FIG. 2).

### Example 4

### 6× His-SUMO-UBA2 Binding Mono- and Polyubiquitin

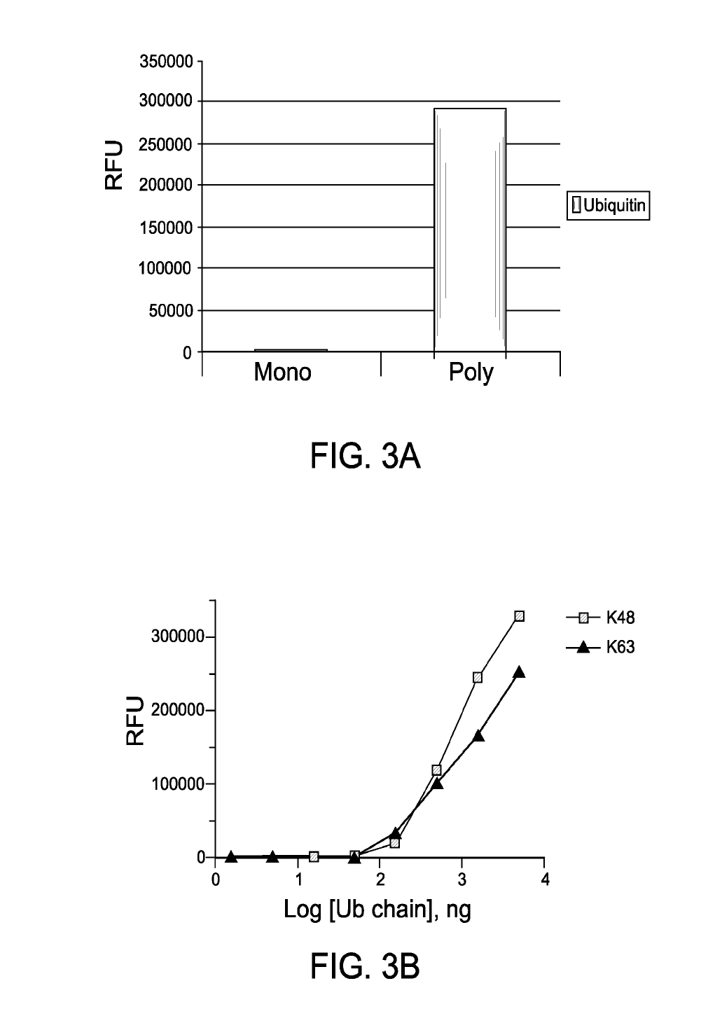

High binding modular plates were coated with 100 μl of 0.1 mg/ml solution of 6× His-SUMO-UBA2 overnight at 4° C. The plate was then incubated with 3% BSA in 1× PBS for 3 hours at room temperature and washed with 1× PBS three times. 30 μl of 0.5 μg ubiquitin and 0.5 μg of K48 polyubiquitin chains (BIOMOL) were transferred to the 6× His-SUMO-UBA2-coated plates and incubated for 2 hours. The wells were washed 3 times with 1× PBS. 100 μl of anti-ubiquitin antibody (1:10 dilution in 0.3% BSA in 1× PBS) was added to the wells and incubated for 1 hour. Plates were washed 3 times with 1× PBS. 100 μl of FITC-labeled anti-rabbit antibody solution (1:50 dilution in 3% BSA in 1× PBS) was added. The plate was incubated at room temperature for 1 hour and the wells were washed 7× with 1× PBS. Using a fluorescence plate reader (Perkin Elmer Envision), fluorescence was detected using excitation and emission wavelengths of 485 nm and 535 nm, respectively. The data in FIG. 3A demonstrate that UBA2-coated plates distinguish monoubiquitin from polyubiquitin.

### Example 5

### 6× His-SUMO-UBA2 Binding K48 and K63 Polyubiquitin Chains

High binding modular plates were coated with 100 μl of 0.1 mg/ml solution of 6× His-SUMO-UBA2 overnight at 4° C. The plates were then incubated with 3% BSA in 1× PBS for 3 hours at room temperature and washed with 1× PBS three times. K48-linked and K63-linked ubiquitin chains (BIOMOL) were added to the wells in concentrations ranging from 5 μg to 1 ng in 30 μl. After a 2 hour incubation, the wells were washed 3 times with 1× PBS. 100 μl of anti-ubiquitin antibody (1:10 dilution in 0.3% BSA in 1× PBS) was added to the wells and incubated for 1 hour. Plates were washed 3 times with 1× PBS. 100 μl of FITC-labeled anti-rabbit antibody solution (1:50 dilution in 3% BSA in 1× PBS) was added. The plate was incubated at room temperature for 1 hour and the wells were washed 7 times with 1× PBS. Using a fluorescence plate reader (Perkin Elmer Envision), fluorescence was detected using excitation and emission wavelengths of 485 nm and 535 nm, respectively. The data in FIG. 3B demonstrate that UBA2-coated plates exhibited an affinity for K48 and K63 polyubiquitin chains.

### Example 6

### SUMO-MuRF1 Ubiquitylation

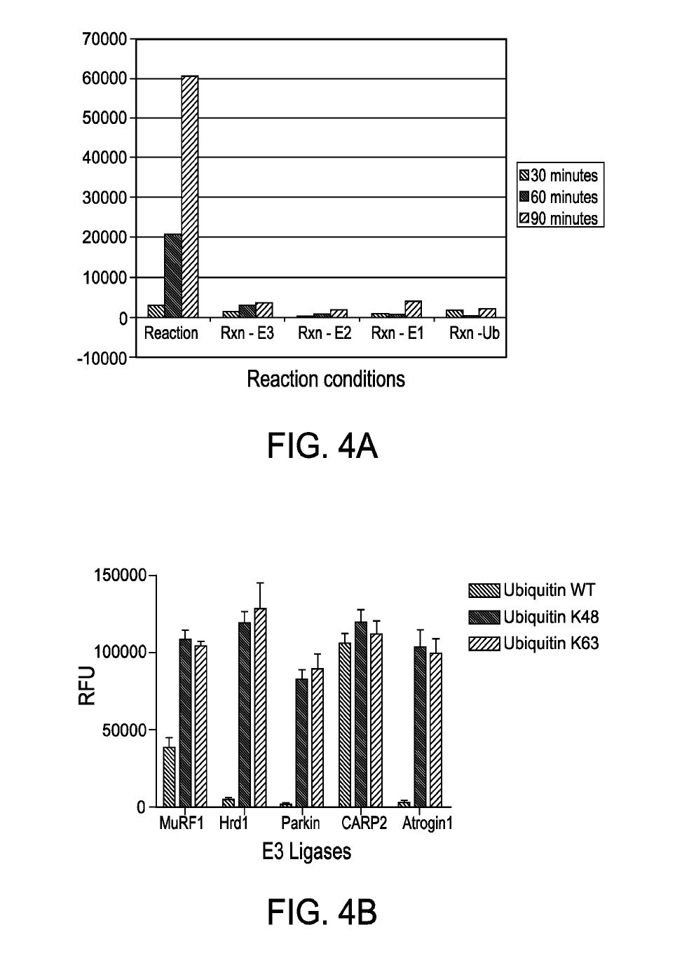

Ubiquitylation reactions contained of 500 nM 6× His-SUMO-MuRF1, 50 nM E1, 150 nM UbcH5c (E2), 500 ng Ubiquitin, 2 mM ATP, 50 mM Tris-HCl pH8.0, 5 mM MgCl₂, 2 mM DTT. Several reactions were conducted where a key component of the reaction was absent (−E3, −E2, −E1, and −Ub). A PCR product encoding MuRF1 was generated using the primers 5′-GATCGGTCTCAAGGTATGGATTATAAGTCGAGCCTG-3′ (SEQ ID NO:12) and 5″-GATCGGTCTCAGATCCATGGATTATAAGTCGAGCCT-3′ (SEQ ID NO:13) and human MuRF1 cDNA as the template. The PCR fragment was digested with BsaI and BamHI, and then ligated into p6× His-SUMO plasmid (LifeSensors) generating 6× His-SUMO-MuRF1. The plasmid was sequenced to confirm the presence of the correct sequence. 30 μl reaction mixtures were incubated for 30, 60, or 90 minutes at 37° C. and transferred to a 6× His-SUMO-UBA2 coated 96-well plate. After a 2 hour incubation, the wells were washed 3 times with 1× PBS. 100 μl of anti-ubiquitin antibody (1:10 dilution in 0.3% BSA in 1× PBS) was added to the wells and incubated for 1 hour. The plate was washed 3 times with 1× PBS. 100 μl of FITC-labeled anti-rabbit antibody solution (1:50 dilution in 3% BSA in 1× PBS) was added. The plate was incubated at room temperature for 1 hour and the wells were washed 7 times with 1× PBS. Using a fluorescence plate reader (Perkin Elmer Envision), fluorescence was detected using excitation and emission wavelengths of 485 nm and 535 nm, respectively. The data in FIG. 4A demonstrate that SUMO-UBA2 detects MuRF 1 autoubiquitylation.

### Example 7

### Ligase Reactions with Various Ubiquitin Substrates

MuRF1, Hrd1, Parkin, CARP2, and Atrogin1 were analyzed using the 6× His-SUMO-UBA2 coated 96-well plate in ubiquitylation experiments with different types of ubiquitin. For the purpose of expression and purification, a truncated Hrd1 protein was used, where the N-terminal trans-membrane domain (encoded by amino acids 1-234) was omitted. A plasmid encoding the truncated protein 6× His-SUMO-Hrd1 Δ235 was constructed as follows. A PCR product was generated using the primers 5′-GATCGGTCTCTAGGTAAGGTGCACACCTTCCCACT-3′(SEQ ID NO:14) and 5′-GATCGGATCCTCAGTGGGCAACAGGAGACT-3′ (SEQ ID NO:15) and human Hrd1 cDNA as the template. The PCR fragment was digested with BsaI and BamHI, and then ligated into p6× His-SUMO plasmid (LifeSensors). The plasmid was sequenced to confirm the presence of the correct sequence. A PCR product encoding Parkin was generated using the primers 5′-GATCCGTCTCAAGGTATGATAGTGTTTGTCAGGTTC-3′ (SEQ ID NO:16) and 5′-GATCGGATCCCTACACGTCGAACCAGTGGTCC-3′ (SEQ ID NO:17) and human Parkin cDNA as the template. The PCR fragment was digested with BsmBI and BamHI, and then ligated into p6× His-SUMO plasmid (LifeSensors) generating 6× His-SUMO-Parkin. The plasmid was sequenced to confirm the presence of the correct sequence. A PCR product encoding Atrogin1 was generated using the primers 5′-GATCGGTCTCAAGGTATGCCATTCCTCGGGCAGGACTG-3′ (SEQ ID NO:18) and 5′-GATCGGATCCTCAGAACTTGAACAAGTTGATAA-3′ (SEQ ID NO:19) and human Atrogin1 cDNA as the template in a PCR. The PCR fragment was digested with BsaI and BamHI, and then ligated into p6× His-SUMO plasmid (LifeSensors) generating 6× His-SUMO-Atrogin1. The plasmid was sequenced to confirm the presence of the correct sequence.

500 nM of each ligase was mixed with 50 nM E1, 150 nM UbcH5c (E2), 500 ng Ubiquitin (either wild-type, K48, or K63), 2 mM ATP, 50 mM Tris-HCl pH8.0, 5 mM MgCl₂, 0.1 mM DTT. A PCR product encoding 6× His-ubiquitin was generated using the primers 5′-GCACCATGGGTCATCACCATCATCATCACGGGCAGATCTTCGTCAGGACG-3′ (SEQ ID NO:20) and 5′-GCAGGATCCGGTCTCAACCTCCACGTAGGCGTAAGAC-3′ (SEQ ID NO:21) and human ubiquitin cDNA as the template in a PCR. The PCR fragment was digested with NcoI and BamHI, and then ligated into pET24D (Novagen). K48 6× His-ubiquitin was generated by PCR using the primers 5′-GCACCATGGGTCATCACCATCATCATCACGGGCAGATCTTCGTCAGGACG-3′ (SEQ ID NO:20) and 5′-GCAGGATCCGGTCTCAACCTCCACGTAGGCGTAAGAC-3′ (SEQ ID NO:21) and a modified human ubiquitin cDNA as the PCR template that had all of the lysine amino acid residues replaced with arginine except lysine 48. The PCR fragment was digested with NcoI and BamHI, and then ligated into pET24D (Novagen). The plasmid was sequenced to confirm the presence of the correct sequence. K63 6× His-ubiquitin was constructed as follows. A PCR product was generated using the primers 5′-GCACCATGGGTCATCACCATCATCATCACGGGCAGATCTTCGTCAGGACG-3′ (SEQ ID NO:20) and 5′-GCAGGATCCGGTCTCAACCTCCACGTAGGCGTAAGAC-3′ (SEQ ID NO:21) and a modified human ubiquitin cDNA as the PCR template that had all of the lysine amino acid residues replaced arginine except for lysine 63. The PCR fragment was digested with NcoI and BamHI, and then ligated into pET24D (Novagen). The plasmid was sequenced to confirm the presence of the correct sequence.

The 30 μl reactions were incubated for 90 minutes at 37° C. in a 6× His-SUMO-UBA2 coated 96-well plate. The wells were washed 3 times with 1× PBS. 100 μl of anti-ubiquitin antibody (1:10 dilution in 0.3% BSA in 1× PBS) was added and incubated for 1 hour. The wells were washed 3 times with 1× PBS. 100 μl of FITC-labeled anti-rabbit antibody solution (1:50 dilution in 3% BSA in 1× PBS) was added and incubated at room temperature for 1 hour. The wells were washed 7 times with 1× PBS. Using a fluorescence plate reader (Perkin Elmer Envision), fluorescence was detected using excitation and emission wavelengths of 485 nm and 535 nm, respectively. The data in FIG. 4B demonstrate that all the ligases tested are active in this assay format.

### Example 8

### SUMO-CARP2 Concentration Dependence

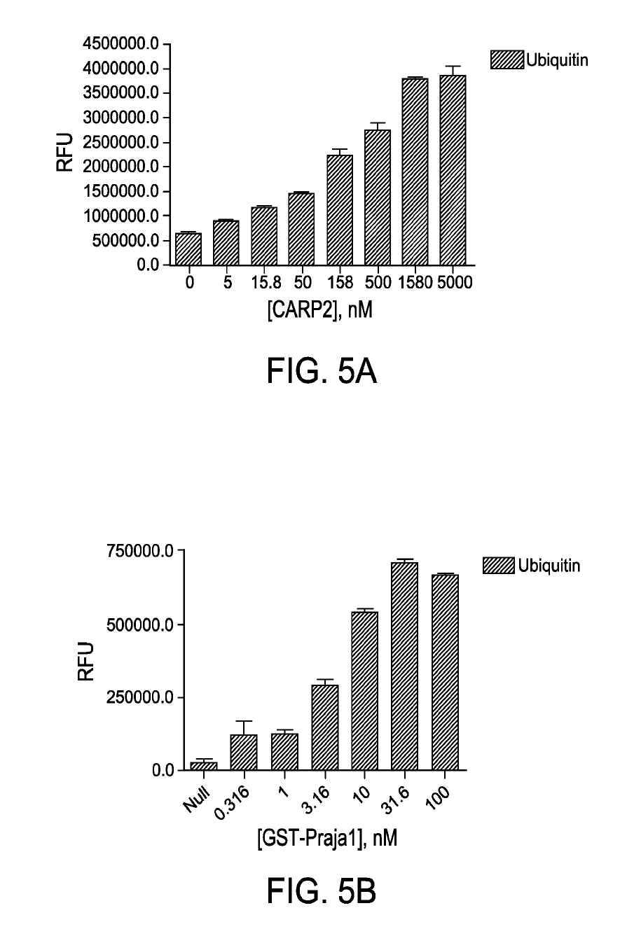

A concentration range of SUMO-CARP2 (0-5 μM) was included in 50 μl ubiquitylation reactions. Ubiquitylation reactions contained 6× His-SUMO-CARP2, 10 nM E1, 100 nM UbcH5c (E2), 500 ng Ubiquitin, 2 mM ATP, 50 mM Tris-HCl pH8.0, 5 mM MgCl₂, 0.1 mM DTT. 50 μl reactions were incubated for 90 minutes at 37° C. in 6× His-SUMO-UBA2 coated 96-well plate. Wells were washed 3 times with 1× PBS. 50 μl of anti-ubiquitin antibody (1:10 dilution in 0.3% BSA in 1× PBS) were added to the wells and incubated for 1 hour. The plate was washed 3 times with 1× PBS. 50 μl of FITC-labeled anti-rabbit antibody solution (1:100 dilution in 3% BSA in 1× PBS) was added. The plate was incubated at room temperature for 1 hour and the wells were washed 4 times with 1× PBS. Using a fluorescence plate reader (Perkin Elmer Envision), fluorescence was detected using excitation and emission wavelengths of 485 nm and 535 nm, respectively. The data in FIG. 5A demonstrate that SUMO-CARP2 autoubiquitylation is concentration dependent.

### Example 9

### GST-Praja1 Concentration Dependence

A concentration range of GST-Praja1 (0-100 nM) was included in 50 μl ubiquitylation reactions. A PCR product encoding Praja1 was generated using the primers 5′-GATCGGATCCCCATGGGTCAGGAATCTAGCAAG-3′ (SEQ ID NO:22) and 5′-GATCGAATTCAGAGTGGGGGAGGGAACATGC-3′ (SEQ ID NO:23) and human Praja1 cDNA (Open Biosystems) as the template. The PCR fragment was digested with BamHI and EcoRI, and then ligated into pGEX3X plasmid (Pharmacia Biotech) generating GST-Praja1. The plasmid was sequenced to confirm the presence of the correct sequence. Ubiquitylation reactions consisted of GST-Praja1, 10 nM E1, 100 nM UbcH5c (E2), 500 ng Ubiquitin, 2 mM ATP, 50 mM Tris-HCl pH8.0, 5 mM MgCl₂, 0.1 mM DTT. The 50 μl reactions were incubated for 90 minutes at 37° C. in 6× His-SUMO-UBA2 coated 96-well plate. The wells were washed 3 times with 1× PBS. 100 μl of anti-ubiquitin antibody (1:10 dilution in 0.3% BSA in 1× PBS) was added to the wells and incubated for 1 hour. Plates were washed 3 times with 1× PBS. 100 μl of FITC-labeled anti-rabbit antibody solution (1:50 dilution in 3% BSA in 1× PBS) were added and plates incubated at room temperature for 1 hour followed by 7 washes with 1× PBS. Using a fluorescence plate reader (Perkin Elmer Envision), fluorescence was detected using excitation and emission wavelengths of 485 nm and 535 nm, respectively. The data in FIG. 5B demonstrate that GST-Praja1 autoubiquitylation is concentration dependent.

### Example 10

### E3 Ligase Assay

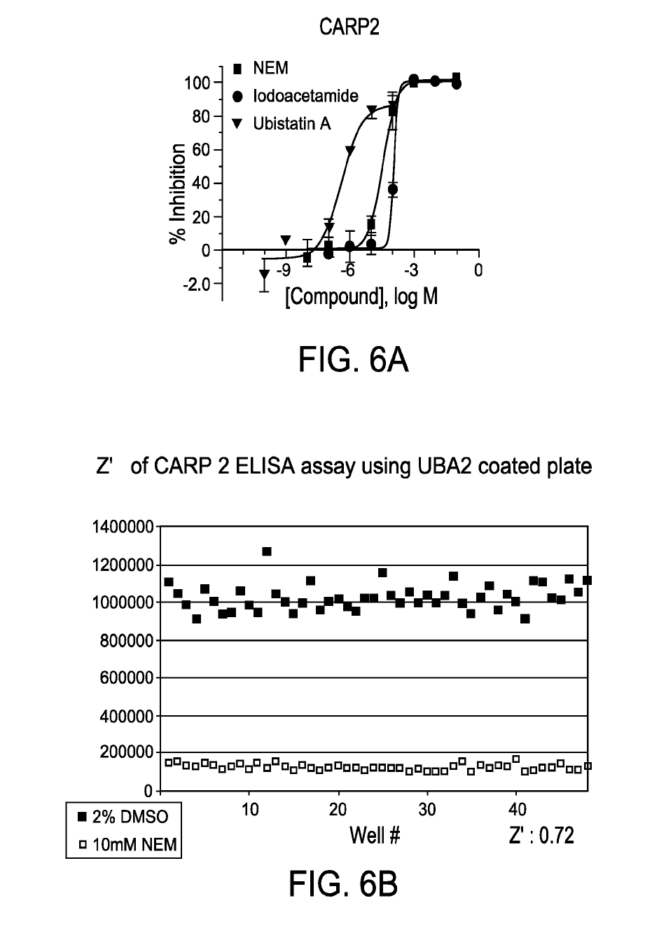

Medium binding plates (Costar, USA) were coated with 100 μl of 0.1 mg/ml solution of 6× His-SUMO-UBA2 overnight at 4° C. The plates were blocked with 200 μl of 3% BSA in 1× phosphate buffered saline (1× PBS, pH 7.4) for 3 hours at 4° C. and washed with 1× PBS three times. E3 reactions were carried out as follows. For IC₅₀ experiments, various concentrations of NEM, iodoacetamide, and ubistatin A were added to individual wells in triplicate. Controls contained 5 μl of 10% DMSO or 50 mM NEM. The compounds were supplemented with 20 μl of enzyme mixture containing GST-E1 (10 nM)/His6-UbcH5c (100 nM), 6× His SUMO-CARP2 (500 nM) prepared in assay buffer (50 mM Tris-HCl, pH8.0, 2 mM MgCl2, 0.1 mM DTT) and incubated at room temperature for 30 minutes. A pFastBac-GST-E1 vector was expressed and purified from insect cells according to published methods. Beaudenon and Huibregtse, Methods Enzymol, 398: 3-8 9 (2005). A PCR product encoding UbcH5c was generated using the primers 5′-GATCTCTAGAATGGCGCTGAAACGGATTAA-3′ (SEQ ID NO:24) and 5′-GATCCTCGAGTCACATGGCATACTTTCTGAGTC-3′ (SEQ ID NO:25) and human UbcH5c cDNA as the template. The PCR fragment was digested with BsaI and BamHI, and then ligated into pET24D (Novagen) generating 6× His-UbcH5c. The plasmid was sequenced to confirm the presence of the correct sequence. E3 reactions were initiated by the addition of 25 μl of ubiquitin (500 ng)/ATP (2 mM) mixture prepared in assay buffer and incubating for 60 minutes at 37° C. Plates were washed with 1× PBS three times and incubated for 1 hour at room temperature with 100 μl of 1:10 dilution of anti-ubiquitin primary antibody (SIGMA, USA) prepared in 0.3% BSA in 1× PBS. Plates were washed with 1× PBS three times followed by incubation with 100 μl of 1:100 dilution of anti-rabbit IgG-FITC conjugate (Jackson ImmunoResearch, USA) for 1 hour at room temperature. Plates were washed six times with 1× PBS and readings were taken using the fluorescence plate reader with excitation and emission wavelengths of 485 nm and 535 nm, respectively. As shown in FIG. 6A, NEM, iodoacetamide, and ubistatinA inhibit the E1, E2 and E3 coupled enzyme reaction with IC₅₀ values of 3.42×10⁻⁵M, 1.1×10⁻⁴M and 4.21×10⁻⁷M, respectively.

For determining the Z′ of the 6× His-SUMO-CARP2 E3 assay, the reaction was carried out as described above except that in wells A1-H6 of 96 well plate, the enzyme mixture was pre-incubated with 10 mM NEM in 2% DMSO, and wells A7-H12 contained 2% DMSO as vehicle control. Data was exported to Excel and Z′ was calculated as described in Zhang et al., J. Biomol. Screen., 1999. 4(2): p. 67-73. The data in FIG. 6B demonstrate that, using UBA2 coated plates the 6× His-SUMO-CARP2 E3 assay gave a Z′ of 0.72.

### Example 11

### 5′-Iodoacetamido Fluorescein Labeling

6× His-SUMO^{G2C}-UBA1 was labeled with 5-Iodoacetamido Fluorescein (“5′-IAF”) by adding 900 μl of 10 mM 5′-IAF solution prepared in dimethylformamide to 2 ml of 8.4 mg of 6× His-SUMO^{G2C}-UBA1 protein in 20 mM Tris-HCl pH 7.46 and 150 mM NaCl in a 15 ml tube and incubating in the dark at 4° C. for ˜15 hours with gentle rotation. 6× His-SUMO-CARP2 was labeled as described above but the reaction was allowed to proceed only for 1 hour at 4° C. The unreacted free 5′-IAF was removed by passing the sample through a PD-10 desalting column equilibrated with 20 mM Tris-HCl pH 7.46 and 150 mM NaCl and eluted with 3 ml buffer. Eluted fractions were analyzed by SDS-PAGE and visualized on a fluorescence gel imager followed by staining with coomassie brilliant blue. The data in FIGS. 7C and 7D show the UBA1 labeling with 5′-IAF and purification respectively.

### Example 12

### SUMO-GFP-CARP2 E3 Ligase Assay

High binding plates were coated with 100 μl of 0.1 mg/ml solution of 6× His-SUMO^{G2C}-UBA1 overnight at 4° C. The plate was incubated with 200 μl of 3% BSA in 1× phosphate buffered saline (1× PBS, pH 7.4) for 3 hours at room temperature and washed with 1× PBS three times. The E3 ligase reaction was assembled in tubes containing 500 nM or 1 μM 6× His-SUMO-GFP-CARP2 (E3), 50 nM E1, 150 nM UbcH5c (E2), 500 ng ubiquitin, 2 mM ATP, 50 mM Tris-HCl pH8.0, 5 mM MgCl2, 0.1 mM DTT. A PCR product encoding CARP2 was generated using the primers 5′-GACGAGCTGTACAAGATGTGGGCAACCTGCTGCAACTGG-3′ (SEQ ID NO:26) and 5′-GTGGTGCTCGAGTCAGGACCGGAAGACATGCACAGCTCG-3′ (SEQ ID NO:27) and pSUMO-CARP2 as the template. The PCR fragment was digested with BsrGI and XhoI and then ligated into pET-6× His-SUMO-GFP plasmid (LifeSensors) generating SUMO-GFP-CARP2 vector. The plasmid was sequenced to confirm the presence of the correct sequence. 30 μl of reaction mixture in triplicate was transferred to the plate containing UBA1-coated wells and incubated at 37° C. for 90 minutes. The wells were washed with 1× PBS and readings were taken using a fluorescence plate reader with excitation and emission wavelengths of 485 nm and 535 nm, respectively. The data in FIG. 7B demonstrate that UBA1 can be used to detect polyubiquitylation of 6× His-GFP-CARP2.

### Example 13

### E3 Ligase Assay Using 5′-IAF-UBA1

E3 ligase reactions contained 10 nM GST-Praja1, 500 nM 6× His-SUMO-CARP2 or 500 nM 6× His-SUMO-MuRF1 as E3s, 50 nM E1, 150 nM UbcH5c (E2), 500 ng Ubiquitin, 2 mM ATP, 50 mM Tris-HCl pH8.0, 5 mM MgCl2, 0.1 mM DTT. For measuring SCF^{Atrogin-1} E3 ligase activity, the reaction was assembled essentially as described above except that the E3 ligase contained 6× His-SUMO-Atrogin-1, Cullin-1 (“Cul1”), Roc-1 (“Rbx-1”) and 6× His-Skp1 (250 nM each). In order to obtain large amounts of the Cul1-Rbx1 complex for the SCF complex, we used a procedure to overexpress the Cul1-Rbx1 complex in E. coli. Zheng, et al., Nature, 416(6882):703-9 (2002). The Cul1 gene was split into two halves (residues 1-410 and residues 411-776) and co-expressed with GST-tagged Rbx1 as three different polypeptide chains. A PCR product encoding Skp1 was generated using the primers 5′-GATCGGTCTCAAGGTATGCCTTCAATTAAGTTGCACAGTTCTGAT-3′ (SEQ ID NO:28) and 5′-GATCGGATCCTCACTTCTCTTCACACCA-3′ (SEQ ID NO:29) and human Skp1 cDNA as the template. The PCR fragment was digested with BsaI and BamHI, and then ligated into pET24D (Novagen). The plasmid was sequenced to confirm the presence of the correct sequence.

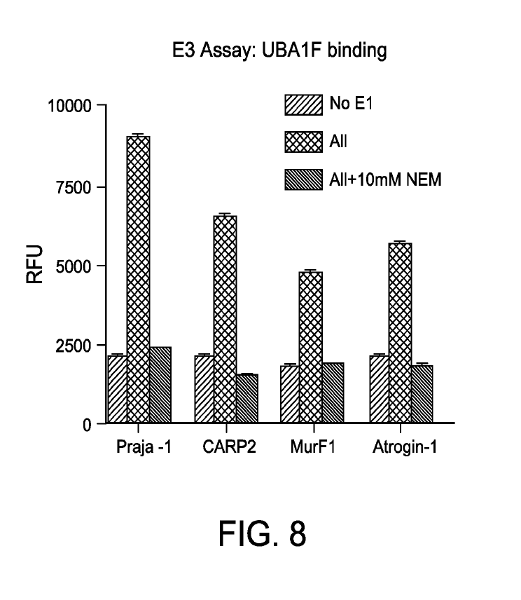

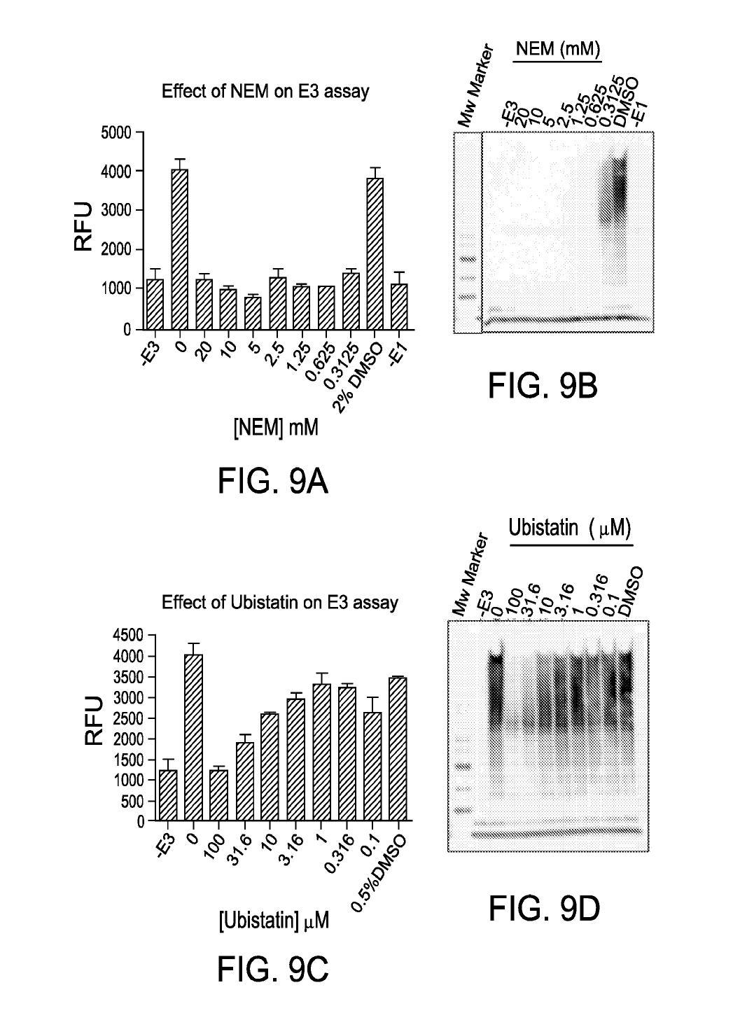

For inhibition assays, reactions containing E1, E2, and E3 were pre-incubated with various concentrations of NEM and ubistatin A for 30 minutes at room temperature before initiating the reaction by adding ubiquitin and ATP. As controls, the reaction was also carried out without adding E3 or E1. The 30 μl reaction mixtures were transferred to the wells of detachable strips and incubated for 90 minutes at 37° C. and then incubated with 3% BSA in 1× PBS for 3 hours. After washing the wells with 1× PBS, the wells were incubated with 100 μl of 5′-IAF-UBA1 for 1 hour. The wells were washed with 1× PBS and readings were taken using a fluorescence plate reader with excitation and emission wavelengths of 485 nm and 535 nm, respectively. Aliquots of the reaction were also separated on SDS-PAGE and immunoblotted with anti-ubiquitin antibody (SIGMA). The data in FIG. 8 demonstrate that 5′-IAF-labeled UBA1 can be used to detect polyubiquitylation of Praja-1, CARP2, MuRF1, and SCF^{Atrogin-1} E3 ligases. The data in FIGS. 9A, B and 9C, D demonstrate that this assay can be inhibited by NEM and Ubistatin A, respectively.

### Example 14

### E3 Ligase Assay Using 5′-IAF-Labeled 6× His-SUMO-CARP2

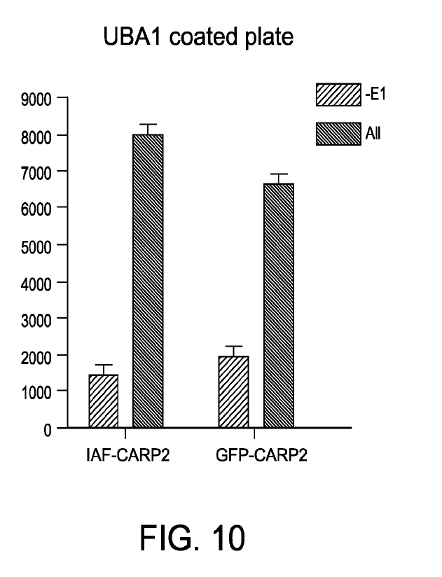

E3 assays were carried out as described previously except that reaction with 5′-IAF-labeled 6× His-SUMO-CARP2 was carried out with 6× His-SUMO^{G2C}-UBA1 coated plates. The data in FIG. 10 demonstrate that UBA1 can be used to detect polyubiquitylation of 5′-IAF-labeled 6× His-SUMO-CARP2 or 6× His-SUMO-GFP-CARP2.

1. A method of identifying a ubiquitin ligase modulator, the method comprising:
(a) combining ubiquitin ligase, ubiquitin-adhering motif-containing protein, ubiquitin-activating enzyme, ubiquitin-conjugating enzyme, ubiquitin or ubiquitin-like protein (Ub/Ubl), adenosine triphosphate (ATP), and substrate for ubiquitin ligase, in the presence of a candidate modulator;
(b) measuring the amount of Ub/Ubl bound to the substrate; and
(c) comparing the amount of Ub/Ubl bound to the substrate in the presence of the candidate modulator to the amount of Ub/Ubl bound to the substrate in the absence of the candidate modulator, whereby the difference in amount of Ub/Ubl bound to the substrate indicates that the candidate modulator is a ubiquitin ligase modulator.

2. The method of claim 1, wherein a positive difference in amount of Ub/Ubl bound to the substrate indicates that the candidate modulator is a ubiquitin ligase activator.

3. The method of claim 1, wherein a negative difference in the amount of Ub/Ubl bound to the substrate indicates that the candidate modulator is a ubiquitin ligase inhibitor.

4. The method of claim 1, wherein the ubiquitin-adhering motif-containing protein is bound to a solid support.

5. The method of claim 1, wherein the ubiquitin-adhering motif-containing protein comprises at least one motif selected from the group consisting of ubiquitin-associated (UBA), ubiquitin-interacting motif (UIM), coupling of ubiquitin to ER degradation (CUE), Np14 zinc finger (NZF), ubiquitin E2 variant (UEV), GRAM-like ubiquitin-binding in Eap45 (GLUE), Motif Interacting with Ubiquitin (MIU) and GAA and Tom1 domain (GAT).

6. The method of claim 1, wherein the ubiquitin ligase comprises mouse double minute 2 (MDM2), muscle ring-finger protein 1 (MuRF1), synoviolin (Hrd1), Parkin, caspase 8/10-associated RING domain protein 1 (CARP1), caspase 8/10-associated RING domain protein2 (CARP2), muscle atrophy F-box (Atrogin1), seven in absentia homolog 2 (Siah2), β-transducin repeat containing protein (β-TrCP), or Praja1.

7. The method of claim 1, wherein measuring the Ub/Ubl bound to the substrate is accomplished using at least one antibody or ubiquitin-adhering motif-containing protein.

8. The method of claim 1, wherein measuring the Ub bound to the substrate is accomplished using fluorescence polarization, fluorescence intensity, fluorescence resonance transfer, chromogenicity, or luminescence.

9. A method of identifying a ubiquitin ligase modulator, the method comprising:
(a) combining ubiquitin ligase, ubiquitin-adhering motif-containing protein, ubiquitin-activating enzyme, ubiquitin-conjugating enzyme, ubiquitin or ubiquitin-like protein (Ub/Ubl), and adenosine triphosphate (ATP), in the presence of a candidate modulator;
(b) measuring the amount of Ub/Ubl bound to ubiquitin ligase; and
(c) comparing the amount of Ub/Ubl bound to ubiquitin ligase in the presence of the candidate modulator to the amount of ubiquitin bound to ubiquitin ligase in the absence of the candidate modulator, whereby the difference in amount of Ub/Ubl bound to ubiquitin ligase indicates that the candidate modulator is a ubiquitin ligase modulator.

10. The method of claim 9, wherein a positive difference in amount of Ub/Ubl bound to ubiquitin ligase indicates that the candidate modulator is a ubiquitin ligase activator.

11. The method of claim 9, wherein a negative difference in the amount of Ub/Ubl bound in amount of ubiquitin bound to ubiquitin ligase indicates that the candidate modulator is a ubiquitin ligase inhibitor.

12. The method of claim 9, wherein the ubiquitin-adhering motif-containing protein is bound to a solid support.

13. The method of claim 9, wherein the ubiquitin-adhering motif-containing protein comprises at least one motif selected from the group consisting of ubiquitin-associated (UBA), ubiquitin-interacting motif (UIM), coupling of ubiquitin to ER degradation (CUE), Np14 zinc finger (NZF), ubiquitin E2 variant (UEV), GRAM-like ubiquitin-binding in Eap45 (GLUE), Motif Interacting with Ubiquitin (MIU) and GAA and Tom1 domain (GAT).

14. The method of claim 9, wherein the ubiquitin ligase comprises mouse double minute 2 (MDM2), muscle ring-finger protein 1 (MuRF1), synoviolin (Hrd1), Parkin, caspase 8/10-associated RING domain protein 1 (CARP1), caspase 8/10-associated RING domain protein2 (CARP2), muscle atrophy F-box (Atrogin1), seven in absentia homolog 2 (Siah2), β-transducin repeat containing protein (β-TrCP), or Praja1.

15. The method of claim 9, wherein measuring the Ub/Ubl bound to ubiquitin ligase is accomplished using at least one antibody or ubiquitin-adhering motif-containing protein.

16. The method of claim 9, wherein measuring the Ub/Ubl bound to the substrate is accomplished using fluorescence polarization, fluorescence intensity, fluorescence resonance transfer, chromogenicity, or luminescence.

17-55. (canceled)

56. The method of claim 1, wherein step (a) is performed in an array of wells, whereby each well contains a plurality of ubiquitin ligase.

57. The method of claim 56, further comprising:
(d) repeating steps (a) through (c), wherein the plurality of ubiquitin ligases are substituted with a subset of ubiquitin ligases from those wells having a difference in amount of ubiquitin bound in step (c) until the well contains one ubiquitin ligase having a difference in amount of ubiquitin bound in step (c).

58. The method of claim 9, wherein step (a) is performed in an array of wells, whereby each well contains a plurality of ubiquitin ligases.

59. The method of claim 58, further comprising:
(d) repeating steps (a) through (c), wherein the plurality of ubiquitin ligases are substituted with a subset of ubiquitin ligases from those wells having a difference in amount of ubiquitin bound in step (c) until the well contains one ubiquitin ligase having a difference in amount of ubiquitin bound in step (c).

60. The method of claim 1, wherein said ubiquitin ligase is aberrantly expressed in a disease or syndrome.

61. The method of claim 9, wherein said ubiquitin ligase is aberrantly expressed in a disease or syndrome.

62. The method of claim 7, wherein said ubiquitin-adhering motif-containing protein contains more than one ubiquitin-adhering motif.

63. The method of claim 62, wherein said ubiquitin-adhering motifs are the same or different.

64. The method of claim 15, wherein said ubiquitin-adhering motif-containing protein contains more than one ubiquitin-adhering motif.

65. The method of claim 64, wherein said ubiquitin-adhering motifs are the same or different.


SEQUENCE LISTING (31 sequences)

SEQ ID NO: 1 - protein, 76 aa, Homo sapiens [sequence omitted]

SEQ ID NO: 2 - protein, 107 aa, Artificial Sequence
  RING domain consensus sequence
CXXCXXXXXXXXXXXXXXXXXXXXXXXXXXXXXXXXXXXXXXXCXXXHXXXCXXCXXXXXXXXXXXXXXXXXXXXXXXXXXXXXXXXXXXXXXXXXXXXXXXXCXXC

SEQ ID NO: 3 - protein, 29 aa, Artificial Sequence
  NZF domain consensus sequence
XXXXWXCXXCXXXNXXXXXXCXXCXXXXX

SEQ ID NO: 4 - DNA, 35 bp, Artificial Sequence
  Primer
gatcggtctcaaggtgttgactatacccccgaaga

SEQ ID NO: 5 - DNA, 30 bp, Artificial Sequence
  Primer
gatcggatcctcagtcggcatgatcgctga

SEQ ID NO: 6 - DNA, 36 bp, Artificial Sequence
  Primer
gatccgtctcaaggtggattcgtggtgggaaccgag

SEQ ID NO: 7 - DNA, 39 bp, Artificial Sequence
  Primer
gatccgtctcagatcctaattttctggaatacccatcag

SEQ ID NO: 8 - DNA, 39 bp, Artificial Sequence
  Primer
gaaggagatataccatgtgtcatcaccatcatcatcacg

SEQ ID NO: 9 - DNA, 39 bp, Artificial Sequence
  Primer
cgtgatgatgatggtgatgacacatggtatatctccttc

SEQ ID NO: 10 - DNA, 35 bp, Artificial Sequence
  Primer
gatccgtctcaaggtatgtgggcaacctgctgcaa

SEQ ID NO: 11 - DNA, 30 bp, Artificial Sequence
  Primer
gatcggatcctcaggaccggaagacatgca

SEQ ID NO: 12 - DNA, 36 bp, Artificial Sequence
  Primer
gatcggtctcaaggtatggattataagtcgagcctg

SEQ ID NO: 13 - DNA, 36 bp, Artificial Sequence
  Primer
gatcggtctcagatccatggattataagtcgagcct

SEQ ID NO: 14 - DNA, 35 bp, Artificial Sequence
  Primer
gatcggtctctaggtaaggtgcacaccttcccact

SEQ ID NO: 15 - DNA, 30 bp, Artificial Sequence
  Primer
gatcggatcctcagtgggcaacaggagact

SEQ ID NO: 16 - DNA, 36 bp, Artificial Sequence
  Primer
gatccgtctcaaggtatgatagtgtttgtcaggttc

SEQ ID NO: 17 - DNA, 32 bp, Artificial Sequence
  Primer
gatcggatccctacacgtcgaaccagtggtcc

SEQ ID NO: 18 - DNA, 38 bp, Artificial Sequence
  Primer
gatcggtctcaaggtatgccattcctcgggcaggactg

SEQ ID NO: 19 - DNA, 33 bp, Artificial Sequence
  Primer
gatcggatcctcagaacttgaacaagttgataa

SEQ ID NO: 20 - DNA, 50 bp, Artificial Sequence
  Primer
gcaccatgggtcatcaccatcatcatcacgggcagatcttcgtcaggacg

SEQ ID NO: 21 - DNA, 37 bp, Artificial Sequence
  Primer
gcaggatccggtctcaacctccacgtaggcgtaagac

SEQ ID NO: 22 - DNA, 33 bp, Artificial Sequence
  Primer
gatcggatccccatgggtcaggaatctagcaag

SEQ ID NO: 23 - DNA, 31 bp, Artificial Sequence
  Primer
gatcgaattcagagtgggggagggaacatgc

SEQ ID NO: 24 - DNA, 30 bp, Artificial Sequence
  Primer
gatctctagaatggcgctgaaacggattaa

SEQ ID NO: 25 - DNA, 33 bp, Artificial Sequence
  Primer
gatcctcgagtcacatggcatactttctgagtc

SEQ ID NO: 26 - DNA, 39 bp, Artificial Sequence
  Primer
gacgagctgtacaagatgtgggcaacctgctgcaactgg

SEQ ID NO: 27 - DNA, 39 bp, Artificial Sequence
  Primer
gtggtgctcgagtcaggaccggaagacatgcacagctcg

SEQ ID NO: 28 - DNA, 45 bp, Artificial Sequence
  Primer
gatcggtctcaaggtatgccttcaattaagttgcacagttctgat

SEQ ID NO: 29 - DNA, 28 bp, Artificial Sequence
  Primer
gatcggatcctcacttctcttcacacca

SEQ ID NO: 30 - protein, 155 aa, Artificial Sequence
  Synthetic construct
MGHHHHHHGSDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGGVDYTPEDDQAISRLCELGFERDLVIQVYFACDKNEEAAANILFSDHAD

SEQ ID NO: 31 - protein, 156 aa, Artificial Sequence
  Synthetic construct
MCHHHHHHGSDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGGFVVGTERNETIERIMEMGYQREEVERALRAAFNNPDRAVEYLLMGIPEN